(19) United States
(12) Patent Application Publication
(10) Pub. No.: US 2025/0230197 A1
(43) Pub. Date: Jul. 17, 2025

(54) MALARIA VACCINE

(71) Applicant: OXFORD UNIVERSITY INNOVATION LIMITED, Oxford (GB)

(72) Inventors: Adrian V.S. HILL, Oxford (GB); Katie EWER, Oxford (GB)

(21) Appl. No.: 18/791,268

(22) Filed: Jul. 31, 2024

Related U.S. Application Data
(63) Continuation of application No. 16/634,099, filed on Jan. 24, 2020, now abandoned.

(30) Foreign Application Priority Data
Jul. 27, 2017 (GB) 1712092.4
Nov. 6, 2017 (GB) 1718337.7

(51) Int. Cl. C07K 14/02; A61K 9/00; A61K 39/29; C07K 14/445

(52) U.S. Cl. CPC C07K 14/02; A61K 9/0019; C07K 14/445; A61K 39/292

(57) ABSTRACT

The invention relates to a composition comprising a polypeptide comprising, or consisting of, the amino acid sequence of SEQ ID) NO: 1, or a sequence having at least 80%, 85%, 90%, 95%, 98%, or 99% sequence identity to SEQ ID NO: 1 (R21), wherein said polypeptide is in the form of a virus-like particle (VLP), wherein said particle comprises less than 10% free hepatitis B surface antigen protein, for use in the immunisation of a human subject susceptible to Plasmodium falciparum infection, characterised in that said composition is administered in a dosage regimen of at least one dose of 1 μg to 20 μg R21 per administration for a subject at least 18 years old, or at least one dose of 0.5 μg to 10 μg R21 per administration for a subject less than 18 years old. The invention also relates to kits, methods and uses.

### CROSS-REFERENCE TO RELATED APPLICATIONS

This application is a continuation under 35 U.S.C. § 120 of U.S. patent application Ser. No. 16/634,099, filed on Jan. 24, 2020 (abandoned); which is a national stage of PCT/GB2018/052122, filed Jul. 27, 2018; which cited the priority under 35 U.S.C. § 119 of United Kingdom Pat. App. Nos. 1718337.7 (filed Nov. 6, 2017) and 1712092.4 (filed Jul. 27, 2017). The contents of the foregoing applications are incorporated herein in their entireties, although the prosecution histories of the foregoing applications are not incorporated herein unless explicitly so stated.

### FIELD OF THE INVENTION

The invention relates to immunogenic compositions for treatment of or protection from malarial pathogens such as Plasmodium falciparum. In particular, the invention relates to use of such compositions in immunising human subjects in specific dosage regimens.

### BACKGROUND

The significant reduction in the mortality associated with malaria over the last 15 years is threatened by the emergence and spread of artemisinin resistance and vector resistance to insecticides. It remains a global health priority to develop a durable and highly efficacious malaria vaccine. The most advanced malaria vaccine candidate, RTS,S/AS01 has completed Phase III testing in a multicentre study across several African sites and demonstrates low-level (˜30%) efficacy against clinical malaria in children aged 5-17 months after a three dose schedule [1-4]. This efficacy wanes rapidly over time after the first 12 months [5] and there remain safety concerns of this vaccine schedule that need addressing in the planned pilot deployment trials that are due to commence in Africa in 2018 [6, 7]. Therefore, there is a significant need to improve on RTS,S/AS01 to achieve the goals set down by the Malaria Vaccine Technology Roadmap-development of a suitable vaccine with at least 75% durable efficacy against clinical malaria by 2030 [8].

R21 has been developed at the Jenner Institute, University of Oxford. This is an improved RTS,S construct that comprises recombinant particles expressing the central repeat and the C-terminus of the circumsporozoite protein (CSP) fused to HBsAg, but without the excess of unfused HBsAg protein found in RTS,S [9]. We recently showed that R21 adjuvanted with Matrix-M elicited comparable humoral immunogenicity to RTS,S/AS01 at much lower doses and reactogenicity was significantly improved with R21/MM compared to RTS,S/AS01 (Venkatraman et al). These promising results provided the basis for us to assess efficacy in a malaria sporozoite challenge study in malaria-naïve adults.

However, there are problems with the prior art approaches such as the partial short-lived field efficacy of RTS,S/AS01. In addition, it is widely thought in the art that an efficacious malaria vaccine is likely to be a combination of different approaches acting on multiple antigens involved in different stages of its complex life cycle [11]. In this regard, a leading alternative, and potentially complementary strategy is heterologous prime-boost immunization with sequential administration of viral-vectored vaccines chimpanzee adenovirus serotype 63 (ChAd63) and modified vaccinia Ankara (MVA), both encoding ME-TRAP (a multiple epitope string fused to the thrombospondin-related adhesion protein). In addition to eliciting antibody responses, this approach elicits potent T cell responses in adults in the UK, as well as adults and infants in malaria endemic areas, and has an excellent track record of safety and tolerability in these populations [12-16]. The ChAd63-MVA ME-TRAP malaria vaccine strategy has demonstrated durable partial efficacy in a controlled human malaria infection (CHMI) study in the UK [17], and partial efficacy was again evident in a subsequent CHMI study with Pf-infected sporozoites [18]. Moreover, a randomised controlled single-blind trial undertaken in Kenyan male adults showed that vaccination reduced the risk of malaria infection by 67% [19]. A recent clinical trial combined RTS,S/AS01 with ChAd63-MVA ME-TRAP in the same regimen for the first time, and tested efficacy against sporozoite challenge. This study demonstrated that combining these vaccines in the same regimen was not only safe and tolerable, but also highly immunogenic and efficacious [20]. Thus these combined approaches are believed in the art to represent the most promising starting point for continued research effort.

Collins et al. 2017 (Scientific Reports, Volume 7, Article 46621) discloses a pre-clinical mouse model study focussed on enhancing protective immunity to Malaria with a highly immunogenic virus like particle vaccine. The authors disclose R21 particles formed from a single CSP-Hepatitis B surface antigen (HBsAg) fusion protein, in contrast to the well-studied RTS,S vaccine from GSK Vaccines which comprises a four-fold excess of HBsAg monomers in addition to the CSP-HBsAg fusion protein (FIG. 1). Immunogenicity in BALB/c mice is demonstrated at ‘very low’ doses when administered with the adjuvants Abisco-100 and Matrix-M. Sterile protection against transgenic sporozoite challenge is demonstrated. The study is confined to mice. One dose is taught: 0.5 μg R21 per mouse, distributed into the tibialis muscles of both hind limbs of each mouse, formulated with 12 μg Matrix-M in a 100 μl total injection. (Page 12, first paragraph of Collins et al.). This is a ratio of 1:24 of R21:Matrix-M.

A key conclusion drawn by Collins et al. is that combination of R21 with other components is desirable. For example, at page 2, end of fifth paragraph, (immediately before “Results” section), it is stated “Moreover, when evaluating the potential for R21 as part of a multicomponent vaccination strategy, protective efficacy was enhanced by combining R21 in MF59 with PbTRAP-based viral vectors”. This conclusion is emphasised at page 11, first paragraph where it is stated “Moreover, mixing and co-administering R21+MF59 with PbTRAP-based viral vectors resulted in an enhancement of efficacy. This result supports the hypothesis that targeting both the sporozoites and the liver stage parasites with both cellular and humoral responses, utilising two different antigens may be able to overcome any leakiness of a sporozoite vaccine”.

WO 2014/111733 discloses a particle comprising a fusion protein of at least one NANP (SEQ ID NO: 6) repeat, some or all of the C-Terminus of the CS protein from Plasmodium falciparum and a Hepatitis B surface antigen. The disclosure relates to immunogenic compositions for use in eliciting immune responses in particular for the prevention of Malaria. More specifically, the R21 fusion protein is described. The only dose given to mice throughout this document is 0.5 μg R21. The section at page 9, lines 16 to 21 of WO 2014/111733 mentions that particular compositions may have doses comprising between about 1 and about 1000 μg of fusion protein. It is not mentioned what organism this dose is intended for. Single doses or multiple doses are contemplated, for example page 10, first paragraph of WO 2014/111733, page 10, third paragraph of WO 2014/111733. Adjuvants are mentioned in WO 2014/111733. For example, page 7, lines 28 to 29 mentions that the composition may comprise an adjuvant, and that the adjuvant may be Abisco or Matrix-M. This disclosure is repeated at page 8, lines 10 to 14 of WO 2014/111733. There is no disclosure or guidance of how much adjuvant would be effective in this document. The examples in this document do not disclose the amount of adjuvant to be used. The only disclosure of the amount of adjuvant such as Matrix-M to be used is the recurrent single amount of 12 μg Matrix-M for administration to mice, which occurs in the legend to FIGS. 13, 15 and 16. When multiple doses are used, it is disclosed that an interlude of 2 weeks to 4 months between doses may be used (page 10, line 15). Protective efficacy in mice is demonstrated for the specific combination of Matrix-M and R21 disclosed, for example in FIG. 10 of WO 2014/111733.

The present invention seeks to overcome problem(s) associated with the prior art.

### SUMMARY

It is an advantage of the invention that extremely low doses per administration, such as doses of antigen R21 and/or adjuvant Matrix-M per administration, are taught. It is surprising that such excellent efficacy results can be achieved with such exceptionally low doses.

Thus the invention is based on this surprisingly effective dosage regimen.

Thus in one aspect the invention provides a composition comprising
a polypeptide comprising, or consisting of, the amino acid sequence of SEQ ID NO: 1, or a sequence having at least 80%, 85%, 90%, 95%, 98%, or 99% sequence identity to SEQ ID NO: 1 (R21),
for use in the treatment or immunisation, preferably immunisation, of a human subject susceptible to Plasmodium falciparum infection,
characterised in that
said composition is administered in a dosage regimen of at least one dose of 1 μg to 20 μg R21 per administration for a subject at least 18 years old, or at least one dose of 0.5 μg to 10 μg R21 per administration for a subject less than 18 years old.

Suitably said composition is administered in a dosage regimen of at least one dose of 5 μg to 20 μg R21 per administration for a subject at least 18 years old, or at least one dose of 2.5 μg to 10 μg R21 per administration for a subject less than 18 years old.

Suitably said composition is administered in a dosage regimen of at least one dose of 10 μg R21 per administration for a subject at least 18 years old, or at least one dose of 5 μg R21 per administration for a subject less than 18 years old.

Suitably the dosage regimen comprises two doses.

Suitably the dosage regimen comprises three doses.

When the dosage regimen comprises two or more doses, suitably the final dose contains 100% of the amount of R21 of the first dose. In some embodiments suitably the final dose contains 10% to 50% of the amount of R21 of the first dose. More suitably the final dose contains 20% of the amount of R21 of the first dose.

Suitably the composition further comprises adjuvant. Suitably said adjuvant is Matrix-M. Suitably said adjuvant is present in a ratio in the range 1:1 to 1:50 of R21:Matrix-M. More suitably said adjuvant is present in a ratio in the range 1:2 to 1:25 of R21:Matrix-M. More suitably said adjuvant is present in a ratio in the range 1:2 to 1:20 of R21:Matrix-M. More suitably said adjuvant is present in a ratio in the range 1:10 to 1:20 of R21:Matrix-M. Most suitably said adjuvant is present in a ratio in the range 1:5 to 1:10 of R21:Matrix-M.

Suitably said dose comprises 10 to 500 μg adjuvant for a subject at least 18 years old, or 5 to 250 μg adjuvant for a subject less than 18 years old, most suitably wherein said adjuvant is Matrix-M.

Suitably said dose comprises 20 to 200 μg adjuvant for a subject at least 18 years old, or 10 to 100 μg adjuvant for a subject less than 18 years old, most suitably wherein said adjuvant is Matrix-M.

Suitably said dose comprises 100 to 200 μg adjuvant for a subject at least 18 years old, or 50 to 100 μg adjuvant for a subject less than 18 years old, most suitably wherein said adjuvant is Matrix-M.

Most suitably said dose comprises 25 to 50 μg adjuvant for a subject at least 18 years old, or 5 to 50 μg adjuvant for a subject less than 18 years old, most suitably wherein said adjuvant is Matrix-M.

Suitably said dose comprises about 10 μg R21 and about 50 μg adjuvant for a subject at least 18 years old, or comprises about 5 μg R21 and about 25 μg adjuvant for a subject less than 18 years old, most suitably wherein said adjuvant is Matrix-M.

Suitably said dose comprises about 5 μg to 10 μg R21, most suitably about 5 μg R21, and about 50 μg adjuvant for a subject less than 18 years old, most suitably wherein said adjuvant is Matrix-M.

Suitably said dose comprises about 2 μg R21 and about 50 μg adjuvant for a subject at least 18 years old, most suitably wherein said adjuvant is Matrix-M. We have generated clinical data (in adults) showing that this dose works well.

When the dosage regimen comprises two or more doses, suitably said doses are administered to said subject at interval(s) of 1 week to 12 weeks, more suitably 3 weeks to 12 weeks. In one embodiment suitably said doses are administered to said subject at interval(s) of 1 to 2 weeks. Most suitably said doses are administered to said subject at an interval of 4 weeks.

In one aspect, the invention relates to a composition as described above, further comprising a polypeptide comprising, or consisting of, the amino acid sequence of SEQ ID NO: 3, or a sequence having at least 80%, 85%, 90%, 95%, 98%, or 99% sequence identity to SEQ ID NO: 3 (Rv21).

In one aspect, the invention relates to a composition as described above, further comprising a viral vector, said viral vector comprising nucleic acid encoding at least one epitope from a malarial antigen, preferably from a P. falciparum or P. vivax antigen.

In one aspect, the invention relates to a composition as described above, wherein said composition is a pharmaceutical composition.

In one aspect, the invention relates to a composition as described above, wherein said composition is a vaccine composition.

In one aspect, the invention relates to a composition as described above, wherein said composition is capable of inducing a protective immune response against P. falciparum in a human.

In one aspect, the invention relates to a kit comprising at least a first and a final composition, said first composition comprising 1 μg to 20 μg R21 per administration for a subject at least 18 years old, or 0.5 μg to 10 μg R21 per administration for a subject less than 18 years old, said composition further comprising adjuvant, wherein said adjuvant is Matrix-M, wherein said adjuvant is present in a ratio in the range 1:1 to 1:50 of R21:Matrix-M;
said final composition comprising 10% to 100%, preferably 10% to 50%, most preferably 20%, of the amount of R21 of the first composition per administration, said final composition further comprising adjuvant, wherein said adjuvant is Matrix-M, wherein said adjuvant is present in a ratio in the range 1:1 to 1:50 of R21:Matrix-M; and
instructions for administration to a human subject.

For the avoidance of doubt, a kit comprising at least a first and a final composition must contain at least two compositions (one first and one final).

Suitably said kit further comprises a second composition, said second composition being identical to said first composition.

For the avoidance of doubt, in this context a kit further comprising a second composition must contain at least three compositions (one first and one second and one final). In other words, suitably said kit comprises three compositions, a first composition and a final composition as described above, and a second composition,
said second composition being identical to said first composition.

In one aspect, the invention relates to use of a composition comprising a polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) in the preparation of a medicament for treatment/immunisation of a human subject susceptible to Plasmodium falciparum infection,
characterised in that
said composition comprises at least one dose of 1 μg to 20 μg R21 per administration for a subject at least 18 years old, or at least one dose of 0.5 μg to 10 μg R21 per administration for a subject less than 18 years old.

Suitably said composition further comprises an adjuvant, wherein said adjuvant is Matrix-M, and wherein said adjuvant is present in a ratio in the range 1:1 to 1:50 of R21:Matrix-M.

In one aspect, the invention relates to a method of immunisation of a human subject susceptible to Plasmodium falciparum infection comprising administering a composition comprising polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) to said subject, wherein said composition is administered in a dosage regimen of at least one dose of 1 μg to 20 μg R21 per administration for a subject at least 18 years old, or at least one dose of 0.5 μg to 10 μg R21 per administration for a subject less than 18 years old. Suitably said composition further comprises an adjuvant, wherein said adjuvant is Matrix-M, and wherein said adjuvant is present in a ratio in the range 1:1 to 1:50 of R21:Matrix-M.

In one aspect the invention relates to a method comprising administering a composition comprising polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) to said subject, wherein said composition is administered in a dosage regimen of at least one dose of 1 μg to 20 μg R21 per administration for a subject at least 18 years old, or at least one dose of 0.5 μg to 10 μg R21 per administration for a subject less than 18 years old. Suitably said composition further comprises an adjuvant, wherein said adjuvant is Matrix-M, and wherein said adjuvant is present in a ratio in the range 1:1 to 1:50 of R21:Matrix-M. Suitably said method is a method of immunising a subject such as a human subject susceptible to Plasmodium falciparum infection; suitably said method is a method of treating a subject such as a human subject susceptible to Plasmodium falciparum infection; suitably said method is a method of immunising a subject such as a human subject against malaria/Plasmodium falciparum infection; suitably said method is a method of treating a subject such as a human subject against malaria/Plasmodium falciparum infection.

In one aspect, the invention relates to a composition, kit, use or method as described above wherein said dosage regimen comprises administration of said polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) to said human subject in an amount in the range 0.0000125 to 0.0003333 mg/Kg for a subject at least 18 years old, or 0.00000625 to 0.0001667 mg/Kg for a subject less than 18 years old.

Suitably said administration is intramuscular, subcutaneous or intradermal. Most suitably said administration is intramuscular.

Suitably said administration is by injection.

In a broad aspect the invention provides a composition comprising
a polypeptide comprising, or consisting of, the amino acid sequence of SEQ ID NO: 1, or a sequence having at least 80%, 85%, 90%, 95%, 98%, or 99% sequence identity to SEQ ID NO: 1 (R21),
characterised in that
said composition is provided in at least one dose of 1 μg to 20 μg R21 per administration for a subject at least 18 years old, or at least one dose of 0.5 μg to 10 μg R21 per administration for a subject less than 18 years old.

Suitably the composition further comprises adjuvant. Suitably said adjuvant is Matrix-M. Suitably said adjuvant is present in a ratio in the range 1:1 to 1:50 of R21:Matrix-M. More suitably said adjuvant is present in a ratio in the range 1:2 to 1:20 of R21:Matrix-M. More suitably said adjuvant is present in a ratio in the range 1:5 to 1:10 of R21:Matrix-M.

Suitably said composition is provided in an amount per dose in the range 0.0000125 to 0.0003333 mg/Kg for a subject at least 18 years old, or 0.00000625 to 0.0001667 mg/Kg for a subject less than 18 years old.

In a preferred embodiment the doses in a multiple dose regime contain the same amount of antigen e.g. R21. When the doses contain the same amount of R21, this is sometimes referred to as a ‘non-fractional dose regime’. Most suitably the invention comprises kits, compositions or methods administering having (or more suitably consisting of) three doses of 10, 10, 10 mcg for adults (subject at least 18 years old) or three doses of 5, 5, 5 mcg for children/infants (subject less than 18 years old).

### DETAILED DESCRIPTION

The inventors provide high level efficacy in humans of a next-generation P. falciparum anti-sporozoite vaccine: R21 in Matrix-M™ adjuvant.

It should be noted that Matrix-M adjuvant has no TLR4 ligand (MPA) in it. By contrast, prior art adjuvants such as ASO1 do comprise TLR4 ligands.

In one embodiment 3 doses are given, one dose being given at 0 weeks, one dose being given at 4 weeks, and one dose being given at 8 weeks. This dosage regimen has the advantage that babies are often brought to clinic for immunisations at these time points, in particular in rural Africa, and so by designing a dosage regimen to be compatible with likely availability of subjects for immunisation then an increased likelihood of correct vaccination is achieved.

In one embodiment suitably the dosage regimen comprises three doses, one dose being given at 0 weeks and one dose being given at 4 weeks and one dose being given at 8 weeks.

In one embodiment suitably the dosage regimen comprises two doses, one dose being given at 0 weeks and one dose being given at 4 weeks. This dosage regimen has the advantage of minimising the number of administrations to two.

In one embodiment, suitably the dosage regimen comprises two doses, one dose being given at 0 weeks and one dose being given at 1 week. This dosage regiment has the advantage of being ideally suited for travellers, especially travellers destined for a Malaria region at short notice.

Durability (such as persistence of a protective response over time) is a problem in the art. For example, organisations such as the Gates Foundation are investing into trying to find new adjuvants to improve durability. The present invention provides technical benefits in the area of durability. For example, the compositions of the invention provide higher concentrations of antigen on the surface of the particles. This is achieved using the R21 polypeptide/particle in the composition of the invention.

The inventors have found that, surprisingly, use of a lower dose of 10 micrograms of R21 in adults induced a more durable immune response 3-6 months after immunisation than use of a higher 50 microgram dose (see FIG. 6). This better durability correlates with better induction of T follicular helper cells of the Tfh2 subset and with increased switched memory B cells, suggesting a potential mechanism for how the lower dose provides a different quality of immune response leading to greater durability (see FIGS. 7A, 7B, & 7C). This evidence of greater durability of the key protective anti-CSP antibody response, to the central NANP repeat, by use of a lower dose rather than a standard high dose of R21 complements our main finding that low dose R21 in matrix-M can provide high level efficacy. Together, the efficacy data plus the durability data make a compelling case for use of a lower dose of R21, such as 10 micrograms, herein suitably used with 50 micrograms of matrix-M.

The present invention may provide enhanced avidity of induced antibodies.

It should be noted that in the field of Malaria high antibody titres are beneficial. For example, it may be considered that titres in excess of 100 μg per ml are beneficial for Malaria (contrasted with diseases such as Men B. where titres of only 2 to 3 μg per ml are considered effective).

It is an advantage of the invention that the dosage regimens taught herein reduce or eliminate the Hep. B response. In this regard, as can be seen from the data presented herein, almost no Hep. B response is induced according to the invention. This is advantageous. Moreover, this has the further benefit that a reduction in the Hep. B response means that the relevant response is a higher proportion of the overall immune response. This is a further benefit delivered by the present invention.

In one embodiment, for adult administration, a ratio of antigen (such as R21) to adjuvant (such as Matrix-M) of 1:5 may be used; suitably a dose comprises 10 μg R21 and 50 μg Matrix-M.

In one embodiment for administration to individuals of less than 18 years a ratio of antigen (such as R21) to adjuvant (such as Matrix-M) of 1:10 may be used; suitably a dose for administration to a subject less than 18 years comprises 5 μg R21 and 50 μg Matrix-M.

In one embodiment a composition comprising 2 μg antigen such as R21 and 50 μg adjuvant such as Matrix-M is used (suitably a ratio of 1:25 of antigen:adjuvant). The inventors have found that this dose and ratio is both safe and highly immunogenic in a phase I study (see FIGS. 4 and 8).

The view in the art is that adjuvant causes reactogenicity. Therefore, the teaching in the art is to use high amounts of adjuvant in order to reduce reactogenicity. In contrast, the low amounts of adjuvant taught in the present invention advantageously still produce excellent immune responses and efficacy.

Without wishing to be bound by theory, it is believed that the compositions used in the invention deliver more antigen per μg (e.g. immunogenic epitopes per μg) compared to prior art formulations such as RTS, S. For example, prior art schemes teach use of 50 μg of RTS, S; sometimes three doses of RTS, S are used with each dose comprising 50 μg RTS, S. In contrast, the present invention teaches advantageously lower antigen amounts such as 10 μg. However, even calculating the “equivalent” antigen delivery per μg of protein/particle in the composition, a 50 μg dose of RTS, S might have an “equivalent” antigen delivery value of approximately 15 μg of R21—advantageously, the inventors teach use of even lower amounts of R21 such as 10 μg R21 per dose. In some embodiments, the invention suitably comprises only 2 μg R21 per dose.

In one embodiment the dosage regimen comprises 2 doses, each dose comprising 2 μg R21.

In one embodiment, the dosage regimen comprises 3 doses, each dose comprising 2 μg R21.

### Amounts of Antigen

Amounts of antigen such as a polypeptide comprising, or consisting of, the amino acid sequence of SEQ ID NO: 1, or a sequence having at least 80%, 85%, 90%, 95%, 98%, or 99% sequence identity to SEQ ID NO: 1 (R21), are taught; for example at least one dose of 1 μg to 20 μg R21 per administration for a subject at least 18 years old, or at least one dose of 0.5 μg to 10 μg R21 per administration for a subject less than 18 years old are taught.

With respect to the caselaw of the European patent office, (e.g. T 198/84 and T 279/89):
(a) the selected sub-range is narrow compared to the known range;

WO 2014/111733 contains disclosures at page 9, lines 16 to 21 which mention that particular compositions may have doses comprising between about 1 and about 1000 μg of fusion protein. It is not mentioned what organism these doses are for. The only examples in WO 2014/111733 are mice. In addition, no age limitations are given in WO 2014/111733—the only examples are mice (which can only be weeks old).

In any case, doses taught herein are narrow compared to WO 2014/111733—compare 1 μg to 20 μg R21 per administration for a subject at least 18 years old, or at least one dose of 0.5 μg to 10 μg R21 per administration for a subject less than 18 years old with the disclosure of “between about 1 and about 1000 μg” in WO 2014/111733.

More importantly, there is no overlap at all in the doses taught in mg/Kg for humans—the doses are 2-3 orders of magnitude apart (see below).
(b) the selected sub-range is sufficiently far removed from any specific examples disclosed in the prior art and from the end-points of the known range;

Suitably mice such as adult mice are considered to weigh 20 g. Therefore amounts of components in the doses/compositions as described in the prior art may be expressed in the same ‘mg/Kg’ terms for comparison. By way of example, a dose comprising 0.5 μg R21 for administration to a mouse such as an adult mouse equates to a dose of (0.0005 mg/(20/1000 Kg)=) 0.025 mg/Kg (as in WO 2014/111733).

Suitably adult humans are considered to weigh 60-80 Kg. Therefore amounts of components in the doses/compositions as described may be converted into ‘mg/Kg’ or other units if desired. By way of example, a dose comprising 10 μg R21 for administration to an adult human equates to a dose of (0.01 mg/60 Kg to 0.01 mg/80 Kg=) 0.000167 to 0.000125 mg/Kg.

Thus the ranges of the invention are separated by 2-3 orders of magnitude from those of the prior art.
(c) the selected range is not an arbitrary specimen of the prior art, i.e. not a mere embodiment of the prior art, but another invention (purposive selection, new technical teaching);

As explained herein, the incredibly and unexpectedly low doses taught herein are surprisingly effective and bring other technical benefits as explained herein and as evidenced by the data included.

Mice typically weigh about 20 gms; humans typically weigh about 60 to 80 kg. Therefore, humans are about 3000 to 4000 times larger than mice. Scaling up the disclosed doses of Collins et al. 2017 (Scientific Reports, Volume 7, Article 46621) of 0.5 μg R21 per mouse for mice to humans would result in a dose of approximately 1500 to 2000 μg R21, with 36,000 to 48,000 μg of Matrix-M, in a volume of 300,000 μl (300 ml). In sharp contrast, the present invention teaches use of only 1 to 20 μg R21 per administration for adult humans (or 0.5 to 10 μg R21 per administration for infants or children). Thus, the inventors surprisingly teach effective doses 2 to 3 orders of magnitude lower than might be contemplated from a consideration of prior art such as Collins et al.

Thus, it is clear that the present invention discloses a new, small, narrow and specific range of effective amounts of R21 useful in compositions for immunising against Malaria. The range is extremely small. A technical effect is specifically associated with this newly disclosed range. The range occupies only approximately 2% of the range disclosed in WO 2014/111733 (1 to 20 μg compared to 1 to 1000 μg). The amounts disclosed herein are taught for humans, whereas the art is focussed on mouse studies. Moreover, specific amounts of Matrix-M are disclosed which are also different from those disclosed in the prior art and also contribute technical effect compared to the prior art. These surprising benefits are discussed in more detail herein. Moreover, although WO 2014/111733 discloses a large range which might be considered to overlap with the range in the present invention, only a single value (0.5 μg R21) is actually demonstrated in this document. Whether or not the document provides an enabling disclosure for the whole of the breadth of the 1 to 1000 μg range of amounts of R21 is not apparent to the skilled person.

### R21 Antigen

The antigen is suitably a polypeptide.

FIG. 1A shows RTS,S. Produced in S. cerevisiae; Highly immunogenic for both CSP repeat and HBsAg; Completed Phase III trial; Efficacy<50% in field trials.

FIG. 1B shows R21. Produced in P. pastoris; Very high immunogenicity for CSP repeat; Non-immunogenic for HBsAg; 100% efficacy with transgenic parasite challenge in mice; Phase I/II trials (matrix-M, AS01).

The polypeptide is suitably R21. The technical details for preparation and manufacture of R21 are as in WO2014/111733 unless otherwise stated herein. The process of preparation and manufacture may be modified by those skilled in the art of generation of virus-like particle vaccines from Pichia.

The R21 polypeptide is suitably assembled into virus-like particles (VLPs). The R21 polypeptide self-assembles—no additional helper protein is required. Thus sometimes the polypeptide may be referred to as a virus-like particle (VLP) or ‘particle’.

In more detail, the antigen may be a particle comprising a fusion protein comprising at least one NANP repeat, some or all of the C-terminus of the CS protein from Plasmodium falciparum and a hepatitis B surface antigen. Preferably the hepatitis B surface antigen is the S antigen. The NANP repeat is a repeat of the four amino acids asparagine, alanine, asparagine, proline which occurs naturally in the CS protein from Plasmodium falciparum. There may be 1, 2, 3, 4, 5, 6, 7, 8, 9, 10, 11, 12, 13, 14, 15, 16, 17, 18 or more repeats of NANP. Preferably there are at least 3 repeats, more preferably there are at least 10 repeats. The fusion protein may in one embodiment comprise 18 repeats of NANP.
“some or all of the C-terminus of the CS protein” has its natural meaning; suitably the fusion protein comprises at least part of the C-terminus of the CS protein from Plasmodium falciparum.

The C-terminus of the CS protein is often referred to as the T-cell epitope containing C-terminus. The C-terminus of the CS protein included in the fusion protein of the invention may comprise the sequence (SEQ ID NO: 7):

NKNNQGNGQGHNMPNDPNRNVDENANANSAVKNNNNEEPSDKHIKEYLNK
IQNSLSTEWSPCSVTCGNGIQVRIKPGSANKPKDELDYANDIEKKICKME
KCSSVFNVVNSSIGI

with some of the C-terminal amino acids deleted. Preferably up to 15 amino acids are deleted, more preferably up to 10 amino acids, 9 amino acids, 8 amino acid, 7 amino acids, 6 amino acids, 5 amino acids, 4 amino acids, 3 amino acids are deleted.

The C-terminus of the CS protein in the fusion protein may have the sequence (SEQ ID NO: 8):

NKN QGNGQGHNMPNDPNRNVDENANANSAVKN N
EEPSDKHIKEYLNKIQNSLST
EWSPCSVTCGNGIQVRIKPGSANKPKDELDYANDIEKKICKMEKCSSV.

The antigenic particle is sometimes referred to as a virus-like particle. It is considered that such particles are more immunogenic than monomeric proteins. Suitably the particle may comprise no, or substantially no, other proteinaceous material. Suitably the particle may comprise no, or substantially no, free hepatitis B surface antigen protein: that is no, or substantially no, hepatitis B surface antigen protein which is not part of the fusion protein. The particle of the invention may comprise no, or substantially no, free CS protein: that is no, or substantially no, CS protein which is not part of the fusion protein.

Reference herein to “substantially no” suitably requires the particle to comprise less than about 10%, 9%, 8%, 7%, 6%, 5%, 4%, 3%, 2% or about 1% of the particular material referred to. Preferably the particles contain less than 5%, more preferably less than 1%, free hepatitis B surface antigen protein.

### Ratios; Differences to RTS

Both R21 and RTS,S are VLPs. These VLPs self-assemble from the polypeptides.

The sequence of the fusion proteins used in R21 and RTS is very similar. There is no change at the N-terminal region or in the repeats of the CS protein or in the HBsAg sequence. There is a truncation at the end of the C-terminus of CSP in R21 compared to RTS.

The main difference between R21 and RTS,S is that the RTS,S has a CSP:HBsAg ratio in the region of 1:5 (i.e. 1:1 for each molecule of RTS fusion protein, but each molecule of RTS fusion protein is accompanied by approx. 4 unfused HBsAg molecules making 1:5 for CSP:HBsAg overall in RTS,S). It is important to note that every polypeptide molecule in the VLP of R21 has CSP sequence, whereas in contrast only one in five molecules in the VLP of RTS,S has CSP sequence. In other words the ratio of CSP sequence to HBsAg sequence in R21 is 1:1, whereas the ratio of CSP sequence to HBsAg sequence in RTS,S is 1:5. This results in a much higher level of exposure of the CSP sequences on R21 than RTS,S.

Regarding sequence differences, below is a comparison of the sequences of R21 and RTS.

-R21 (410 aa's)
Seq ID No: 1
MDPNANPNANPNANPNANPNANPNANPNANPNANPNANPNANPN
ANPNANPNANPNANPNANPNANPNANPNANPNKNNQGNGQGHNM
PNDPNRNVDENANANSAVKNNNNEEPSDKHIKEYLNKIQNSLST
EWSPCSVTCGNGIQVRIKPGSANKPKDELDYANDIEKKICKMEK
CSSVPVTNMENITSGFLGPLLVLQAGFFLLTRILTIPQSLDSWW
TSLNFLGGSPVCLGQNSQSPTSNHSPTSCPPICPGYRWMCLRRF
IIFLFILLLCLIFLLVLLDYQGMLPVCPLIPGSTTTNTGPCKTC
TTPAQGNSMFPSCCCTKPTDGNCTCIPIPSSWAFAKYLWEWASV
RFSWLSLLVPFVQWFVGLSPTVWLSAIWMMWYWGPSLYSIVSPF
IPLLPIFFCLWVYI
-RTSS (11 C-terminal amino acids more plus 3
extra at N-terminus)(424 aa's)
Seq ID No: 5
MMAPDPNANPNANPNANPNANPNANPNANPNANPNANPNANPNA
NPNANPNANPNANPNANPNANPNANPNANPNANPNKNNQGNGQG
HNMPNDPNRNVDENANANSAVKNNNNEEPSDKHIKEYLNKIQNS
LSTEWSPCSVTCGNGIQVRIKPGSANKPKDELDYANDIEKKICK
MEKCSSVFNVVNSSIGLGPVTNMENITSGFLGPLLVLQAGFFLL
TRILT1PQSLDSWWTSLNFLGGSPVCLGQNSQS PTSNHSPTSC
PPICPGYRWMCLRRFIIFLFILLLCLIFLLVLLDYQGMLPVCPL
IPGSTTTNTGPCKTCTTPAQGNSMFPSCCCTKPTDGNCTCIPIP
SSWAFAKYLWEWASVRFSWLSLLVPFVQWFVGLSPTVWLSAIWM
MWYWGPSLYSIVSPFIPLLPIFFCLWVYI

R21 410aa compared to RTSS 424aa=96.7% identical
C-Terminal region of circumsporozoite protein underlined in sequences above
R21=105 amino acids
RTS,S=116 amino acids
Therefore, 90.5% identity across CSP sequence.
Overall sequence identity R21 to RTSS=96.7% identical.

Considering only the CSP sequence (C-terminal region of the circumsporozoite) (circumsporozoite protein C-terminal region) sequence identity R21 to RTSS=90.5% identical.

Suitably the composition, kit, use or method of the invention comprises a polypeptide having at least 97% sequence identity to SEQ ID NO: 1.

Suitably the composition, kit, use or method of the invention comprises a polypeptide having a CSP sequence having at least 91% sequence identity to the CSP sequence of SEQ ID NO: 1.

In any case, a key advantage of R21 over RTS,S derives from the fact that R21 VLPs advantageously avoid (i.e. have an absence of) the four-fold excess of hepatitis B surface antigen (“S”) which is found in RTS,S VLPs. In other words suitably the ratio of CSP:HsBAg is 1:1 in R21, so that the R21 vaccine does not “waste” its immunogenicity by making really strong immune responses to hepatitis B, which is irrelevant to malaria prevention. Thus an advantage of R21 over RTS,S is that the immune responses are concentrated more fully on the malarial epitopes, rather than being ‘diluted’ by the excess of HsBAg epitopes found in RTS,S.

Suitably the composition of the invention comprises polypeptide, the polypeptide is present as VLP, and the VLP comprises CSP sequence and HsBAg sequence, wherein the ratio of CSP:HsBAg in the VLP is 1:1.

In a preferred embodiment suitably the polypeptide is present in the form of a virus-like particle (VLP), and the VLP comprises parts of the central repeat and the C-terminus of the circumsporozoite protein (CSP) sequence and the Hepatitis B surface antigen (HBsAg) sequence as a fusion protein but without any unfused hepatitis B surface protein molecules in the VLP. This has the further advantage of the absence of unfused HBsAg in the VLP which is the striking difference from RTS,S. The phrase “parts of” refers to the fact that R21 does not have the full central repeat or C-terminal sequence. R21 has about half the number of NANP central repeats as in common malaria strains, i.e. 19 rather than 40, and R21 has truncated the C-terminal region at its end by 20 amino acids.

Suitably the composition of the invention comprises polypeptide, wherein said polypeptide is present in the form of a virus-like particle (VLP), and wherein the VLP comprises the circumsporozoite protein (CSP) sequence and the Hepatitis B surface antigen (HBsAg) sequence in a 1:1 ratio.

Suitably the composition of the invention comprises polypeptide, wherein said polypeptide is present in the form of a virus-like particle (VLP), and wherein the VLP comprises the C-terminus of the circumsporozoite protein (CSP) sequence and the Hepatitis B surface antigen (HBsAg) sequence in a 1:1 ratio.

Suitably the composition of the invention comprises polypeptide, wherein said polypeptide is present in the form of a virus-like particle (VLP), and wherein the VLP comprises the central repeat and the C-terminus of the circumsporozoite protein (CSP) sequence and the Hepatitis B surface antigen (HBsAg) sequence in a 1:1 ratio.

Thus in one aspect the invention provides a composition comprising
a polypeptide,
wherein said polypeptide comprises, or consists of, the amino acid sequence of SEQ ID NO: 1, or a sequence having at least 80%, 85%, 90%, 95%, 98%, or 99% sequence identity to SEQ ID NO: 1 (R21),
wherein said polypeptide is in the form of a virus-like particle (VLP), and wherein the VLP comprises circumsporozoite protein (CSP) sequence and Hepatitis B surface antigen (HBsAg) sequence in a 1:1 ratio, for use in the immunisation of a human subject susceptible to Plasmodium falciparum infection,
characterised in that
said composition is administered in a dosage regimen of at least one dose of 1 μg to 20 μg R21 per administration for a subject at least 18 years old, or at least one dose of 0.5 μg to 10 μg R21 per administration for a subject less than 18 years old.

Thus in one aspect the invention provides a composition comprising
a polypeptide comprising, or consisting of, the amino acid sequence of SEQ ID NO: 1, or a sequence having at least 80%, 85%, 90%, 95%, 98%, or 99% sequence identity to SEQ ID NO: 1 (R21),
   wherein said polypeptide is in the form of a virus-like particle (VLP), wherein said particle comprises less than 10% free hepatitis B surface antigen protein,
for use in the immunisation of a human subject susceptible to Plasmodium falciparum infection,
characterised in that
said composition is administered in a dosage regimen of at least one dose of 1 μg to 20 μg R21 per administration for a subject at least 18 years old, or at least one dose of 0.5 μg to 10 μg R21 per administration for a subject less than 18 years old.
   Thus in one aspect the invention provides a method of immunisation of a human subject susceptible to Plasmodium falciparum infection comprising administering a composition comprising polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) wherein said polypeptide is in the form of a virus-like particle (VLP), wherein said particle comprises less than 10% free hepatitis B surface antigen protein,
      to said subject, wherein said composition is administered in a dosage regimen of at least one dose of 1 μg to 20 μg R21 per administration for a subject at least 18 years old, or at least one dose of 0.5 μg to 10 μg R21 per administration for a subject less than 18 years old.

Thus in one aspect the invention provides a composition, kit, use or method according to any preceding claim wherein said dosage regimen comprises administration of said polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) wherein said polypeptide is in the form of a virus-like particle (VLP), wherein said particle comprises less than 10% free hepatitis B surface antigen protein,

to said human subject in an amount in the range 0.0000125 to 0.0003333 mg/Kg for a subject at least 18 years old, or 0.00000625 to 0.001667 mg/Kg for a subject less than 18 years old.

Thus in one aspect the invention provides a method of immunisation of a human subject susceptible to Plasmodium falciparum infection comprising administering a composition comprising polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) wherein said polypeptide is in the form of a virus-like particle (VLP), and wherein the VLP comprises circumsporozoite protein (CSP) sequence and Hepatitis B surface antigen (HBsAg) sequence in a 1:1 ratio,
to said subject, wherein said composition is administered in a dosage regimen of at least one dose of 1 μg to 20 μg R21 per administration for a subject at least 18 years old, or at least one dose of 0.5 μg to 10 μg R21 per administration for a subject less than 18 years old.

Thus in one aspect the invention provides a composition, kit, use or method according to any preceding claim wherein said dosage regimen comprises administration of said polypeptide comprising, or consisting of, the amino acid sequence of SEQ ID NO: 1, or a sequence having at least 80%, 85%, 90%, 95%, 98%, or 99% sequence identity to SEQ ID NO: 1 (R21) wherein said polypeptide is in the form of a virus-like particle (VLP), and wherein the VLP comprises circumsporozoite protein (CSP) sequence and Hepatitis B surface antigen (HBsAg) sequence in a 1:1 ratio,
to said human subject in an amount in the range 0.0000125 to 0.0003333 mg/Kg for a subject at least 18 years old, or 0.00000625 to 0.001667 mg/Kg for a subject less than 18 years old.

Preferably, at least about 40% or more by mass of the proteinaceous material of the particle is derived from Plasmodium falciparum. The ability to have such a high level of Plasmodium falciparum material in the particles allows a more favourable antibody response with respect to malaria, more specifically a significant antibody response to Plasmodium falciparum and a smaller antibody response to the hepatitis B surface antigen.

A reduction in the relative amount of hepatitis B surface antigen in the particles may also have the advantage that the particles have improved efficacy in early infancy. If too much hepatitis B surface antigen is present there is concern that maternal antibodies present in a young infant may make the particles less effective as immunogens.

Preferably in a fusion protein of the invention the hepatitis B surface antigen is C-terminal to any Plasmodium falciparum material.

The particle may comprise a fusion protein comprising, or consisting of, the sequence of SEQ ID NO: 1 (R21) or a sequence with at least 80%, 85%, 90%, 95%, 98%, 99% or more sequence identity with the sequence of SEQ ID NO: 1.

Percentage sequence identity is defined as the percentage of amino acids in a sequence that are identical with the amino acids in a provided sequence after aligning the sequences and introducing gaps if necessary to achieve the maximum percent sequence identity. Alignment for the purpose of determining percent sequence identity can be achieved in many ways that are well known to the man skilled in the art, and include, for example, using BLAST (National Center for Biotechnology Information Basic Local Alignment Search Tool).

Variations in percent identity may be due, for example, to amino acid substitutions, insertions or deletions. Amino acid substitutions may be conservative in nature, in that the substituted amino acid has similar structural and/or chemical properties, for example the substitution of leucine with isoleucine is a conservative substitution.

Preferably a polypeptide includes sequences with conservative substitutions which do not have any significant effect on the immunogenicity of the resulting fusion protein.

Conservative substitutions may be made, for example according to the Table below. Amino acids in the same block in the second column and suitably in the same line in the third column may be substituted for each other:

| | | |
|---|---|---|
| ALIPHATIC | Non-polar | Gly Ala Pro |
| | | Ile Leu Val |
| | Polar - uncharged | Cys Ser Thr Met |
| | | Asn Gly |
| | Polar - charged | Asp Glu |
| | | Lys Arg |
| AROMATIC | | His Phe TrpTyr |

Substitutions may also be introduced to match better the CS sequence of other strains of Plasmodium falciparum. The sequence used in the R21 example reported here is of the 3D7 strain.

Preferably a particle comprises numerous monomers of the fusion protein. The particle may comprise a least 10 fusion protein monomers, preferably 20 or more, 30 or more, 40 or more, 50 or more, 60 or more, 70 or more, 80 or more, 90 or more, 100 or more fusion protein monomers. In one embodiment the particle comprises around 96 fusion protein monomers.

Preferably the particle is immunogenic. A particle is suitably capable of eliciting an immune response against the malaria causing parasite Plasmodium falciparum. The immune response may be therapeutic and/or prophylactic. The immune response may be sufficient to reduce or prevent infection or disease cause by Plasmodium falciparum. The particle may elicit/produce a protective immune response when administered to a subject, preferably a human subject.

Preferably the immune response elicited by the composition of the invention affects the ability of Plasmodium falciparum to infect an immunised human. Preferably the ability of Plasmodium falciparum to infect a human immunised with the composition of the invention is impeded or prevented. This may be achieved in a number of ways. The immune response elicited may recognise and destroy Plasmodium falciparum. Alternatively, or additionally, the immune response elicited may impede or prevent replication of Plasmodium falciparum. Alternatively, or additionally, the immune response elicited may impede or prevent Plasmodium falciparum causing disease in the subject such as a human. Preferably the immune response elicited is an antibody response.

Suitably the subject is a human.

The composition may be provided in a liquid formulation. Alternatively, the composition may be provided in a lyophilised form. Alternatively the composition may be provided in a sugar based formulation dried on membranes as described by Alcock et al. (Sci Transl Med. 2010 Feb. 17; 2 (19): 19ral2).

Polypeptide such as R21 as particles may be produced expressing the fusion protein in Saccharomyces cerevisiae or Pichia pastoris or another methylotrophic yeast such as Hansenula polymorpha and recovering the fusion protein, preferably in the form of particles.

If the fusion protein is expressed in Pichia pastoris, or another methylotrophic yeast, expression of the protein may be driven by the AOXI promoter or by the GAP promoter or by another strong promoter (Vogl & Glieder, New Biotechnology. 2012 Nov. 16. pii: S 1871-6784 (12) 00867-9).

If the fusion protein is expressed in Saccharomyces cerevisiae, expression of the protein may be driven by the TDH3 promoter or by another strong promoter.

Preferably the fusion protein is expressed at sufficiently high levels that upon lysis of the yeast the fusion proteins spontaneously multimerise to form particles, sometime referred to as virus like particles (VLPs).

A nucleic acid, such as DNA, encoding the fusion protein may be transiently or constitutively expressed by the yeast. The nucleic acid encoding the fusion protein may be integrated into the host genome or may be carried on an extracellular component, such as a plasmid. The yeast may contain, 1, 2, 3, 4, 5 or more copies of the nucleic acid encoding the fusion protein.

The nucleic acid encoding the fusion protein may be codon optimised for expression in yeast.

A person skilled in the art would be readily able to prepare a suitable host to express the nucleic acid encoding the fusion protein.

Preferably the Saccharomyces cerevisiae or Pichia pastoris or another methylotrophic yeast used in the method of the invention does not express any, or any significant, hepatitis B surface antigen protein which is not part of the fusion protein.

Preferably the Saccharomyces cerevisiae or Pichia pastoris or another methylotrophic yeast used in the method of the invention does not express any, or any significant, CS protein from Plasmodium falciparum which is not part of the fusion protein.

The ability to express particles according to the invention in a high yielding yeast strain, such as Pichia pastoris, may simplify and enhance the biomanufacture of the polypeptide leading to lower cost of goods for manufacture. This saving in cost is particularly important for a malaria vaccine which is targeted primarily at populations, especially children and infants, in low income countries who require a low cost vaccine.

A nucleic acid sequence encoding a the polypeptide may be synthesised. A vector containing the nucleic acid sequence, wherein the nucleic acid sequence may be operably linked to transcriptional control elements, may be constructed

The composition may be a pharmaceutical composition.

The composition may be a vaccine composition.

The composition is suitably for use in the prevention of malaria.

The composition may comprise a pharmaceutically acceptable carrier, diluent or excipient.

Suitable acceptable excipients and carriers will be well known to those skilled in the art. These may include solid or liquid carriers. Suitable liquid carriers include water and saline. The polypeptide of the composition may be formulated into an emulsion or may be formulated into biodegradable microspheres or liposomes.

The composition may also comprise polymers or other agents to control the consistency of the composition, and/or to control the release of the antigen/polypeptide from the composition.

Diluents may include water, saline, glycerol or other suitable alcohols etc.

The composition may comprise further constituents such as wetting or emulsifying agents; buffering agents; thickening agents for example cellulose or cellulose derivatives; preservatives; detergents, antimicrobial agents; and the like.

Preferably the active ingredients in the composition are greater than 50% pure, usually greater than 80% pure, often greater than 90% pure and more preferably greater than 95%, 98% or 99% pure. With active ingredients approaching 100% pure, for example about 99.5% pure or about 99.9% pure, being most suitable.

The composition of the invention may also include in admixture one or more further antigens. The one or more further antigens may be derived from Plasmodium falciparum or from other species of Plasmodium, such as Plasmodium vivax or Plasmodium malariae.

The pharmaceutical composition or vaccine composition may be provided in a liquid form or in a lyophilised form.

Preferably the pharmaceutical composition or vaccine composition is capable of producing a protective immune response to Plasmodium falciparum.

The phrase “producing a protective immune response” as used herein means that the composition is capable of generating a protective response in a host organism, such as a human mammal, to whom it is administered. Preferably a protective immune response protects against subsequent infection or disease caused by Plasmodium falciparum. The protective immune response may eliminate or reduce the level of infection by reducing replication of Plasmodium falciparum or by affecting the mode of action of Plasmodium falciparum to reduce disease.

Preferably, if the composition is used as a vaccine, the composition comprises an immunologically effective amount of polypeptide according to the invention. An “immunologically effective amount” of an antigen is an amount that when administered to an individual, according to the regimen taught herein, is effective for treatment or prevention of infection by Plasmodium falciparum. This amount will vary depending upon the health and physical condition of the individual to be treated and on the antigen. Precise amounts are disclosed as part of the regimens discussed herein.

The composition may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

More suitably the composition is for intramuscular, subcutaneous or intradermal administration.

Most suitably the composition is for intramuscular administration.

Most suitably the composition is for injection.

Thus suitably administration is intramuscular, subcutaneous or intradermal.

Most suitably administration is intramuscular.

Most suitably administration is by injection.

Compositions of the invention may be able to induce serum antibody responses which mediate the destruction or inactivation of the Plasmodium falciparum after being administered to a subject. The compositions of the invention may also, or alternatively, be able to elicit an immune response which neutralises Plasmodium falciparum, thereby preventing them from having their normal function and preventing or reducing disease progression without necessarily destroying the Plasmodium falciparum.

A composition according to the invention may be used in isolation, or it may be combined with one or more other immunogenic or vaccine compositions, and/or with one or more other therapeutic regimes.

Suitably the R21 fusion protein is used as in WO2014/111733. A most preferred example is given in SEQ ID NO: 1. For some of the supporting data herein, this R21 was used but with a small, 4 amino acid C-terminal extension, known as a “C-tag” which allows easier immunochromatographic purification of the protein particle. Thus R21 may optionally have a C-tag (EPEA) sequence at the C-terminus. This is sometimes referred to as “R21c”—see SEQ ID NO: 2. R21c has the 4 amino acid C-terminal extension: EPEA (glutamic acid-proline-glutamic acid-alanine). Suitably for human use the non-C-tagged version of R21 is used (SEQ ID NO: 1) or a polypeptide having high sequence identity thereto as specified below.

### Adjuvants

The composition may further comprise an adjuvant. The adjuvant may contain saponin. The adjuvant may be Abisco, or matrix M.

The adjuvant may be a squalene-based adjuvant and/or an ISCOM-based adjuvant, such as Abisco/Matrix M (from Isconova, Uppsala—now ‘Novavax AB’).

Abisco-100 (known as Matrix-M when made to GMP standard) has the following chemical content: purified saponins obtained from a crude extract of the plant Quillaja saponaria Molina; cholesterol from Lanolin and phosphatidyl choline (phospholipid) from fresh egg yolk; in a suspension of nano-sized (40 nm) cage-like particles consisting of the above ingredients, in PBS.

Matrix M (or Abisco-100) consists of a mixture of Matrix A and Matrix C at a ratio of 80:20 to 95:5, preferably 85:15. Matrix A leads to T cell induction and has low toxicity, Matrix C induces antibodies and has some toxicity. Matrix C contains C fraction of QS separation which corresponds to QS21. Fraction A (in Matrix A) corresponds to QS7.

Abisco-100 and Matrix-M are pre-clinical and clinical versions of the same adjuvant from Novavax AB respectively. Abisco-100 is known as Matrix-M when made to GMP standard. Suitably the adjuvant of the invention is Abisco-100 or Matrix-M.

Most suitably the adjuvant of the invention is Matrix-M, which has the advantage of being clinically acceptable for human use.

Most suitably Matrix-M is from Novavax AB, Kungsgatan, 109, SE-753 18 Uppsala, Sweden.

### Ratios of Components

Suitably the antigen (such as R21) and the adjuvant (such as Matrix-M) are administered, or are present in the composition, in the ratios disclosed herein.

| | Amount | Amount | Amount | Amount |
|---|---|---|---|---|
| Ratio | antigen | adjuvant | antigen | adjuvant |
| antigen:adjuvant | (>=18 years) | (>=18 years) | (<18 years) | (<18 years) |
| 1:1 to 1:50 | 1 μg to 20 μg | (1 μg to 50 μg) | 0.5 μg to 10 μg | (0.5 μg to 25 μg) |
| | | to | | to |
| | | (20 μg to 1000 μg) | | (10 μg to 500 μg) |
| 1:2 to 1:20 | 1 μg to 20 μg | (2 μg to 20 μg) | 0.5 μg to 10 μg | (1 μg to 10 μg) |
| | | to | | to |
| | | (40 μg to 400 μg) | | (20 μg to 200 μg) |
| 1:10 to 1:20 | 1 μg to 20 μg | (10 μg to 200 μg) | 0.5 μg to 10 μg | (5 μg to 10 μg) |
| | | to | | to |
| | | (200 μg to 400 μg) | | (100 μg to 200 μg) |
| 1:25 | 1 μg to 20 μg | (25 μg) to (500 μg) | 0.5 μg to 10 μg | (12.5 μg) to (250 μg) |
| 1:1 to 1:50 | 5 μg to 20 μg | (5 μg to 250 μg) | 2.5 μg to 10 μg | (2.5 μg to 125 μg) |
| | | to | | to |
| | | (20 μg to 1000 μg) | | (10 μg to 500 μg) |
| 1:2 to 1:20 | 5 μg to 20 μg | (10 μg to 100 μg) | 2.5 μg to 10 μg | (5 μg to 50 μg) |
| | | to | | to |
| | | (40 μg to 400 μg) | | (20 μg to 200 μg) |
| 1:10 to 1:20 | 5 μg to 20 μg | (50 μg to 100 μg) | 2.5 μg to 10 μg | (25 μg to 50 μg) |
| | | to | | to |
| | | (200 μg to 400 μg) | | (100 μg to 200 μg) |
| 1:25 | 5 μg to 20 μg | (125 μg) to (500 μg) | 2.5 μg to 10 μg | (62.5 μg) to (250 μg) |
| 1:1 to 1:50 | 10 μg | (10 μg) to (500 μg) | 5 μg | (5 μg) to (250 μg) |
| 1:2 to 1:20 | 10 μg | (20 μg) to (200 μg) | 5 μg | (10 μg) to (100 μg) |
| 1:10 to 1:20 | 10 μg | (100 μg) to (200 μg) | 5 μg | (50 μg) to (100 μg) |
| 1:25 | 10 μg | (250 μg) | 5 μg | (125 μg) |

Unless otherwise apparent from the context, doses mentioned herein are for humans.

Unless otherwise apparent from the context, amounts of components of the compositions mentioned herein are given ‘per dose’. Of course it may be desired to prepare a larger batch of the compositions mentioned, and to divide it or aliquot it into doses later on, for example before administration or before distribution/transportation.

### Doses

A dose is an amount of composition for a single administration to a human subject.

Thus it can be appreciated that a composition of the invention may be provided in an amount containing multiple doses. This is useful for example to minimise costs of packing and distribution—a single phial containing multiple doses may be transported/refrigerated for a lower cost than one dose per phial. Doses may simply be withdrawn at the point of administration. A single phial may contain an amount of composition for the number of doses to be administered. The amount of composition may be ‘overpacked’ to provide a margin for error e.g. if an amount of the composition cannot be withdrawn from the phial due to surface tension, or risk of introducing air bubbles or airlocks during the process of administration. Thus in one embodiment the invention relates to a phial containing at least two doses of composition according to the present invention, more suitably at least three doses of composition according to the present invention, more suitably at least two doses plus 10% of composition according to the present invention, more suitably at least three doses plus 10% of composition according to the present invention.

In some embodiments the doses provided or administered may have different antigen amounts such as different R21 amounts. In this regard, the ‘final’ composition should have its normal meaning i.e. the last composition administered to a subject in a single regimen of immunisation (course of immunisation).

For example, for the first dose the R21 amount will be as described above, for example 1 μg to 20 μg R21 per administration for a subject at least 18 years old, or 0.5 μg to 10 μg R21 per administration for a subject less than 18 years old. For the final dose (i.e. the second dose in a two dose regimen and the third dose in a three dose regime) the R21 amount may be reduced 2-10 fold, most suitably reduced 5 fold. In other words, for the final dose (i.e. the second dose in a two dose regimen or the third dose in a three dose regime) the R21 amount may be 10-50% of the amount in the first dose, most suitably 20% of the amount in the first dose.

In other embodiments the final dose has an R21 amount 100% of the amount of the first does. In other embodiments each dose has the same R21 amount.

Clearly, although amounts in the first dose are conveniently expressed in ranges, the actual amount administered will have an absolute value, depending for example on operator choice, or on the weight of the subject, or on the age of the subject etc. Therefore the amount in the final dose will also have an absolute value by reference to the actual amount administered in the first dose. Suitably the actual amount administered in the first dose is recorded. Suitably the amount in the final dose is calculated by reference to said recorded actual amount administered in the first dose.

By way of example, if the first dose for a subject at least 18 years old comprises 10 μg R21, in one embodiment suitably the final dose comprises 1 to 5 μg R21, most suitably 2 μg R21.

In one embodiment the dosage regimen may comprise 2 doses—a first dose at 10 μg R21, and a final dose at 1 to 5 μg R21, most suitably 2 μg R21.

In one embodiment the dosage regimen may comprise 3 doses—a first dose at 10 μg R21, a second dose identical to the first dose (i.e. a second dose at 10 μg R21) and a final dose at 1 to 5 μg R21, most suitably 2 μg R21.

Suitably kits according to the present invention comprise instructions for administration to a human subject. Suitably said instructions specify one or more of: the dosage amount of antigen, the dosage amount of adjuvant, the number of doses, the interval between doses, and the route of administration, each as described herein.

Suitably said instructions are printed instructions. Suitably said instructions may be printed on a label. Said label may be attached to the container containing the composition.

In one embodiment vaccinations with 10 μg R21/50 μg Matrix M1 are used.

In one embodiment 3 vaccinations with 10 μg R21/50 μg Matrix M1 (including the third at week 8) are used. In one embodiment vaccinations with 10 μg R21/50 μg Matrix M1 at week 8 are used, with the final (e.g. third) dose reduced from 50 mcg to 10 mcg.

In one embodiment a 3 dose regimen is preferred. The 3 dose regime (sometimes referred to as ‘standard regime’) works very well providing 82% efficacy. Most suitably three doses are given at intervals of four weeks.

The inventors have generated evidence from immune responses that two doses may be sufficient. A two dose regime would be highly beneficial in practice and demonstrating good efficacy with two doses represents a breakthrough. Giving only 2 doses saves cost and labour in administration, and additionally facilitates a higher proportion of subjects completing their course of doses.

Doses in mg/Kg

| μg polypeptide | Range (mg/Kg) for human at | |
|---|---|---|
| such as R21 | least 18 years old | |
| μg | from (60 Kg) | to (80 Kg) |
| 1 | 0.0000167 | 0.0000125 |
| 2 | 0.0000333 | 0.000025 |
| 3 | 0.00005 | 0.0000375 |
| 4 | 0.0000667 | 0.00005 |
| 5 | 0.0000833 | 0.0000625 |
| 6 | 0.0001 | 0.000075 |
| 7 | 0.0001167 | 0.0000875 |
| 8 | 0.0001333 | 0.0001 |
| 9 | 0.00015 | 0.0001125 |
| 10 | 0.0001667 | 0.000125 |
| 11 | 0.0001833 | 0.0001375 |
| 12 | 0.0002 | 0.00015 |
| 13 | 0.0002167 | 0.0001625 |
| 14 | 0.0002333 | 0.000175 |
| 15 | 0.00025 | 0.0001875 |
| 16 | 0.0002667 | 0.0002 |
| 17 | 0.0002833 | 0.0002125 |
| 18 | 0.0003 | 0.000225 |
| 19 | 0.0003167 | 0.0002375 |
| 20 | 0.0003333 | 0.00025 |

Doses in mg/Kg

| μg polypeptide | Range (mg/Kg) for human | |
|---|---|---|
| such as R21 | less than 18 years old* | |
| μg | from (6 Kg)* | to (80 Kg)* |
| 0.5 | 0.0000835 | 0.00000625 |
| 1 | 0.000167 | 0.0000125 |
| 2 | 0.000333 | 0.000025 |
| 3 | 0.0005 | 0.0000375 |
| 4 | 0.000667 | 0.00005 |
| 5 | 0.000833 | 0.0000625 |
| 6 | 0.001 | 0.000075 |
| 7 | 0.001167 | 0.0000875 |
| 8 | 0.001333 | 0.0001 |
| 9 | 0.0015 | 0.0001125 |
| 10 | 0.001667 | 0.000125 |

*subjects less than 18 years old of differing ages may have differing weights and the physician will typically take this into account when determining dose. In particular, infants from 2 to 12 months of age may weigh less than the values shown above-the doses in mg/Kg provided herein may simply be used to calculate the corresponding dose taking into account the weight of the subject to which the dose will be administered.

### Intervals

Unless otherwise apparent from the context, the interval is the time between doses. The first dose given is ‘day 0/day zero’. The interval is the time until the next dose.

Suitably an interval of 2 days to 12 months may be used.

More suitably an interval of 1 week to 12 weeks is used.

More suitably an interval of 3 weeks to 12 weeks is used.

Most suitably an interval of 4 weeks is used.

For example, the following vaccine regimens may be used:
Group 1 vaccinations—in week 0, 4 and 8
Group 2 vaccinations—in week 0, 4 and 8
Group 3 vaccinations—in week 0, 4 and 8 in addition to viral-vectored vaccines in week 1 and 9 (or other convenient time points).

More suitably group 3 vaccinations—in week 0, 4 and 8 in addition to viral-vectored vaccines in week 1 and 9.

### Interference

A further advantage may be reducing or avoiding interference problems.

It is desirable to provide protection against both P. falciparum (e.g. by using R21) and P. vivax malaria. Therefore in some embodiments the invention relates to compositions for immunisation against both pathogens. The invention advantageously avoids or reduces interference problems which might be expected with such an approach.

Suitably protection against P. vivax is achieved by administration of Rv21. Suitably Rv21 is as described in Salman et al 2017 (Rational development of a protective P. vivax vaccine evaluated with transgenic rodent parasite challenge models. Sci. Rep. 7, 46482), which is hereby incorporated herein by reference, specifically for the construction of the Rv21 VLP.

In more detail, Rv21 is a virus like particle (VLP) consisting of the chimeric PvCSP VK210/VK247 central repeats and the CSP C-terminal sequence fused to the Hepatitis B Surface Antigen (HepB-S) gene, optionally with a C-terminally placed four amino acid C-tag sequence (Glu-Pro-Glu-Ala). The tag may be omitted or included for human use—e.g. in the clinical trial described in the examples section the tag is included. Most suitably the tag is omitted for human use. Codon usage of the fusion genes was optimized for expression in Pichia pastoris and production of the intracellular fusion protein (PvCSP-HepB-S) was assessed in three protease knockout strains and protease wild-type P. pastoris strain using a time course study expression. The double knock-out P. pastoris strain for prb1 and pep4 proteases had optimal protein expression levels after 108 hours of methanol induction. The presence of the fusion protein PvCSP-HepB S was confirmed by Western blot analyses using antibodies against PvCSP VK210, PvCSP VK247 and HepB S. Presence, size and purity of the Rv21 protein was carried out using a sensitive silver stain technique.

The protocol used for purification of the fusion protein VLP has been used for R21 and involved two steps (Collins, Brod et al. Scientific Reports, 2017). The first consisted of an affinity purification using a capture select C-tag matrix bound to the fusion protein under neutral conditions. In addition to collecting the expected protein band corresponding to the PvCSP-HepB S protein, the sample also contained additional proteins. VLP particle assembly was detected using transmission electron microscopy (TEM). A subsequent purification step was performed by size exclusion chromatography in order to clean up the sample from the other proteins with different molecular size and to remove the high concentration of salts (Elution buffer). A single protein band of expected size of the PvCSP-HepB S protein (75 kDa) was visualized using the silver staining technique and the TEM showed a more homogeneous population of VLPs having a globular shape with some protuberances or spikes on the surface, likely due to the PvCSP protein presence on the surface. The purified Rv21 was used to immunize mice, using a low dose of 0.5 μg/mouse and employing a homologous prime-boost immunization protocol using an interval of one week between immunizations. Rather than administering the ASO1 adjuvant, standardly used for RTS,S vaccination, Matrix-M adjuvant (Novavax AB, Uppsala, Sweden) was used to enhance the immunogenicity and protective efficacy of Rv21. Matrix-M is suitable for human use and consists of saponin-based 40 nm particles that can activate and recruit immune cells to the draining lymph nodes and spleen.

The regimen of the invention administers these, R21 with Rv21, as a mixture (e.g. in 2-3 doses as above).

Advantageously this regimen has the advantage of non-interference (of one with the other).

Thus in one embodiment the invention provides a composition as described above, further comprising Rv21.

When the composition of the invention further comprises Rv21, suitably the amount of Rv21 used should be about the same as for R21, most suitably exactly as for R21.

### Further Embodiments

In one embodiment the dose comprises, or consists of, 10 mcg R21 in 50 mcg matrix-M (for adults) (i.e. ratio R21:Matrix-M=1:5), and/or 5 mcg R21 in 50 mcg matrix-M (for children) (i.e. ratio R21:Matrix-M=1:10).

In one embodiment the dose comprises a constant amount of adjuvant such as Matrix-M. This constant amount may be 50 mcg matrix-M. Thus the invention provides one or more dose(s) comprising, or consisting of, 10 mcg R21 in 50 mcg matrix-M; or 5 mcg R21 in 50 mcg matrix-M; or 2 mcg R21 in 50 mcg matrix-M. The 50 mcg matrix-M amount may be for adults (subject at least 18 years old) and/or for children (subject less than 18 years old). Clearly the amount of antigen such as R21 should still be carefully selected according to the guidance given herein.

### Advantages

Documents such as Salman et al 2017 (Rational development of a protective P. vivax vaccine evaluated with transgenic rodent parasite challenge models. Sci. Rep. 7, 46482), and more relevant documents dealing with P. falciparum/R21 as cited in the background section above, are focussed on mice. Mice are a very poor guide to the dosage required of a vaccine in humans and this must be determined for example in clinical trials. Mice are typically 20 grams and human adults weigh typically 60-80 Kilograms, which is a 3000-4000 fold difference. So a simple extrapolation of a 1 mcgs dose working in a mouse would require 3000-4000 mcgs in a human. However, a better guide is the dose of other similar vaccines used in humans. The most closely related vaccine to R21 is RTS,S for which the standard adult dosage is 50 mcgs. This is why the inventors initially tested 50 mcg in their first two clinical trials (Vac053 and Vac056). However, in subsequent immunisations it was surprisingly found that 1-20 mcg, especially 10 mcg, and even 2 mcg in human adults was a suitable dose of R21. These dosages are 2.5-fold, 5-fold and 25-fold less than that required to produce the same immune response with RTS,S—this is a very surprising finding. Salman et al (ibid.) showed for Rv21 that the required dose for efficacy in 20 gram mice was either 5 mcg or 0.5 mcg per dose, suggesting that the dose in humans would be at least 50 mcg, and likely substantially more, because of the >3000 greater mass of humans.

It should be noted that Collins et al. 2017 ultimately recommends the combination of R21 with viral based vectors such as PbTRAP-based viral vectors. In contrast, the present invention is concerned with the administration of R21, and in particular R21 in extremely low doses (2 to 3 orders of magnitude lower than those taught by Collins et al.) for protective efficacy. This is surprising in itself due to the exceptionally low doses used. Moreover, it is further surprising in view of the teachings of Collins et al. since the present invention shows that the particular doses and administration regimes produce these effects without the need for combination with other vectors such as PbTRAP-based viral vectors, which is a further advantage of the invention.

Mere extrapolation from the prior art is not possible, and the very high immunogenicity and efficacy with the low doses taught herein is genuinely surprising to the inventors. In addition, the strikingly improved safety profile with low doses is another surprising technical benefit and is something that the inventors themselves did not anticipate. Thus a key contribution to the art is the low dose approach (compositions/regimens) taught herein. This approach has utility for manufacture (low costs), safety (less side effects demonstrated) as well as durability of the response: significantly better with low dose (1-20 mcg, most suitably 10 mcg) than full dose (50 mcgs).

In addition, there are specific benefits to embodiments comprising two-dose regimens; protecting human subjects such as infants with two doses rather than three can be a huge benefit for developing country deployment-so a logistic benefit as well as a benefit for reduced cost of goods (i.e. reduced cost of fewer doses), as well as improved safety and durability (which are advantages which the lower dose provides as well).

As noted above, interference with combination(s) is a challenge in the field. In mice we have tested this. The inventors assert that there is enough evidence that there is often interference with mixed subunit vaccines; the approach described herein has the additional benefit of non-interference. In this context the combination is R21 with Rv21.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 shows diagrams. Comparison of some characteristics of R21 and RTS,S vaccine virus-like particles. A greater density of CSP sequences on the surface of the R21 VLP compared to the RTS,S particle may relate to favourable characteristics of the R21 vaccine such as the lack of induction of significant levels of antibodies to the HBsAg sequence.

FIG. 2 shows a flow chart. Vac053 phase I clinical trial of R21 vaccine conducted in the UK with Oxford as the main clinical centre (Venkatraman et al). 75 subjects were screened for eligibility and 31 enrolled. Note that subjects in group 2 did not receive an adjuvant with R21. Doses of R21 as shown in the figure. In groups 1, 3 and 4 the dose of matrix-M was 50 micrograms in all subjects.

FIG. 3 shows a bar chart. Better Safety Profile for R21/Matrix-M 50 μg RTS,S/AS01 vs 10 μg R21/Matrix-M. Safety profile of R21 (10 mcgs) in matrix-M adjuvant (50 mcgs) compared to RTS,S/AS01 (50 mcgs of RTS,S. Local and systemic adverse events were graded on a standard severity scale of 1 (least) to 3 (most severe). The reactogenicity profile of R21/matrix-M was statistically significantly better than RTS,S/AS01 after the second dose (data shown) and also after the first dose (data not shown). After the third dose again the safety profile of R21/matrix-M was better but not significantly so.

FIG. 4, including FIGS. 4A and 4B, show bar charts. Excellent Safety Profile of Low Dose 2 μg R21/Matrix-M is shown. The safety profile of R21/matrix-M was improved further by decreasing the dose of R21 (but not matrix-M) further to just 2 micrograms (FIG. 4B).

FIG. 5, including FIGS. 5A and 5B, shows graphs. R21 Clinical Immunogenicity Data are shown; FIG. 5A shows Immunogenicity dependent on Matrix-M; FIG. 5B shows 10 μg R21/MM comparable with 50 μg RTS,S/AS01B.

FIG. 5A: Median time courses of IgG against the NANP repeat region of CSP in VAC53. Time courses shown are medians for Group 1 (=“G1”) (10 μg R21 in matrix-M (here abbreviated to MM)), G2 (50 μg (=50 micrograms) R21 with no adjuvant) and G3 (50 μg R21 in MM) and are calculated using data for all volunteers that completed follow-up: G1 n=10, G2 n=3, G3 n=8. Median NANP-specific IgG levels were significantly higher after the second and third vaccinations in the adjuvanted groups compared to the groups that received unadjuvanted R21. NANP-specific IgG was boosted again after the third vaccination, but only in groups that received R21 in MM. The highest median antibody response to NANP was observed in the 10 μg R21/MM group 2 weeks after the third vaccination.

FIG. 5B: Mean time courses of NANP-specific IgG are shown for G1 and G3 in VAC53 and compared to responses seen in the VAC55 trial (NCT: 01883609) in volunteers that received 50 μg RTS,S/AS01B at week 0, 4 and 8. Mean NANP-specific IgG levels are comparable between the 10 μg R21/MM and 50 μg RTS,S/AS01B group after each vaccination.

Antibodies responses against NANP are analysed using an enzyme-linked immunosorbent assay (ELISA) and were conducted as published by Rampling et al (J Infect Dis (2016) 214 (5): 772-781. DOI: https://doi.org/10.1093/infdis/jiw244). A pool of serum positive for NANP-specific IgG was used to form a standard curve on each plate. Arbitrary ELISA Units (EUs) were calculated for each sample based on the optical density (OD405) of the sample and the parameters of the standard curve.

FIG. 6 shows plots. Durability of Antibody Response; 10 μg R21 generated significantly higher antibody titres than 50 μg R21 at 6 months.

Durability of the NANP-specific IgG responses measured using the standardised ELISA method used in FIG. 5. NANP-specific IgG was measured for VAC53 G1 and G3 volunteers at day 238 (D238). At this late time point, antibody levels were significantly higher in the 10 μg R21/MM group compared to the 50 μg R21/MM group (Mann-Whitney analysis, P=0.02, lines show medians) indicating surprisingly better durability with the lower dose of vaccine.

FIG. 7, including FIGS. 7A, 7B, and 7C, shows plots. Lower dose regimen induces a qualitatively different Tfh response and increased B cells.

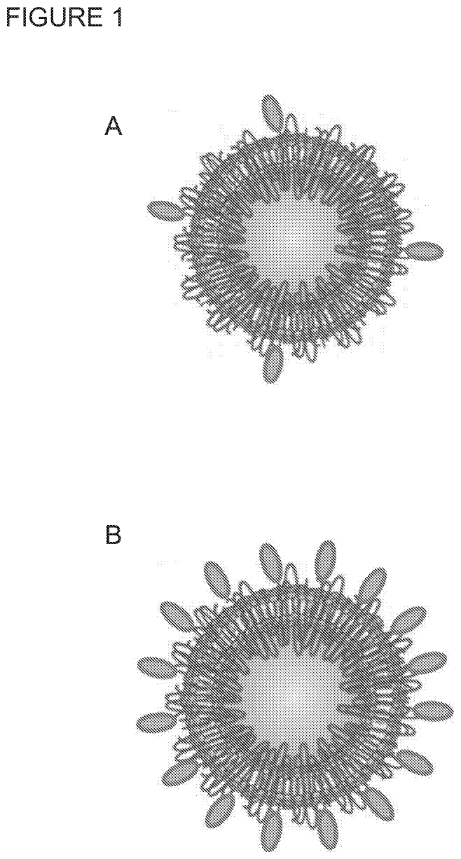

Phenotyping of total circulating T follicular helper cells (cTfh) and B cells in peripheral blood mononucleocytes (PBMC) from VAC65 volunteers at the time point of one day before the malaria challenge, denoted day C−1 (D76). Comparison of 10 μg R21/MM and 50 μg R21/MM. Phenotyping of cTfh is achieved by staining cells with fluorescently labelled antibodies against specific markers and analysed by flow cytometry. Circulating Tfh are defined as single, live, lymphocytes that are PD1+CXCR5+CD45RA−CD4+ T cells and are further divided into subsets by expression of chemokine receptors CXCR3 and CCR6 as: Tfh17 (CXCR3−CCR6+), double positive/dp (CXCR3+CCR6+), Tfh1 (CXCR3+CCR6−) and Tfh2 (CXCR3−CCR6−) as previously published (Schmitt, Bentebibel and Ueno, Trends in Immunology, 2014. DOI: http://dx.doi.org/10.1016/j.it.2014.06.002). B cells were phenotyped in the same manner using markers for CD19, CD20, CD21, CD27, IgD, IgG and IgM. CD19+CD20+ B cells were classified by expression of IgD and CD27 as “switched memory” (IgD-CD27+), “non-switched memory” (IgD+CD27+), “double negative” (IgD−CD27−) or “naïve” (IgD+CD27+) as previously published (Sanz et al Semin Immunol 2008 DOI: 10.1016/j.smim.2007.12.006).

↑ total B cells; ↑ switched memory B cells (IgD-CD27+); ↑ IgG+MBC; ↑ IgG:IgM.

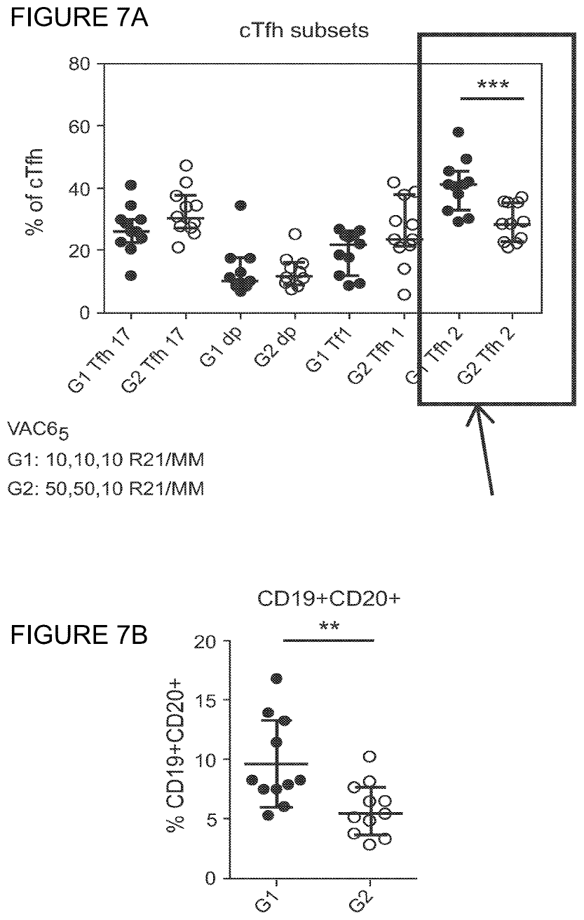

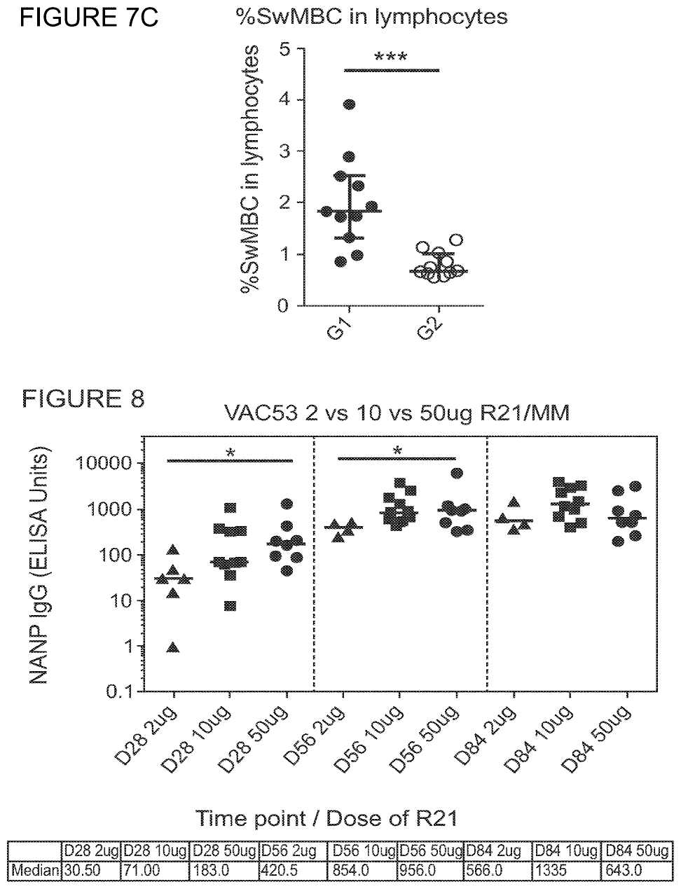

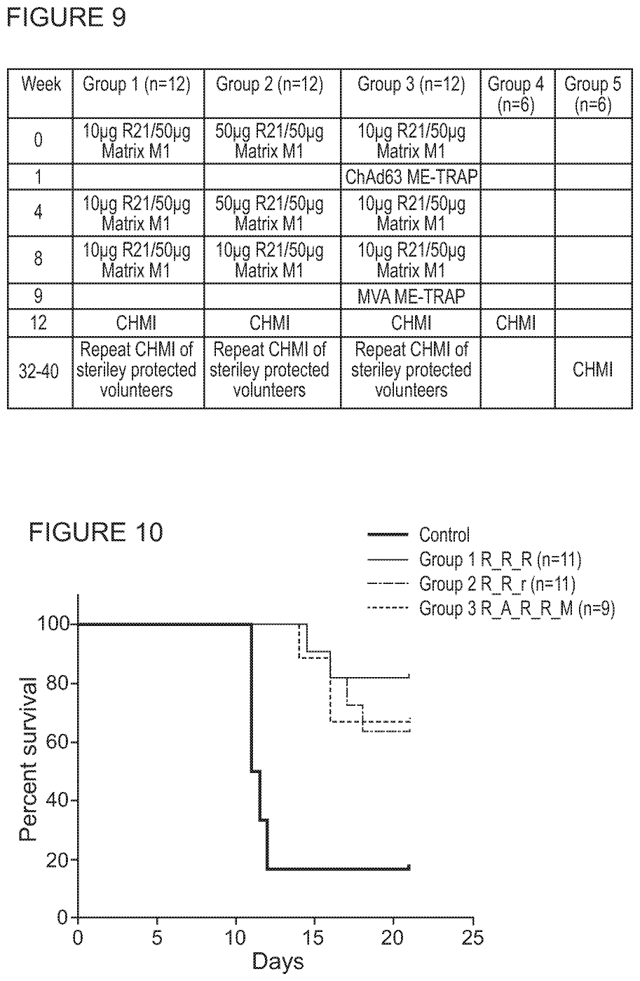

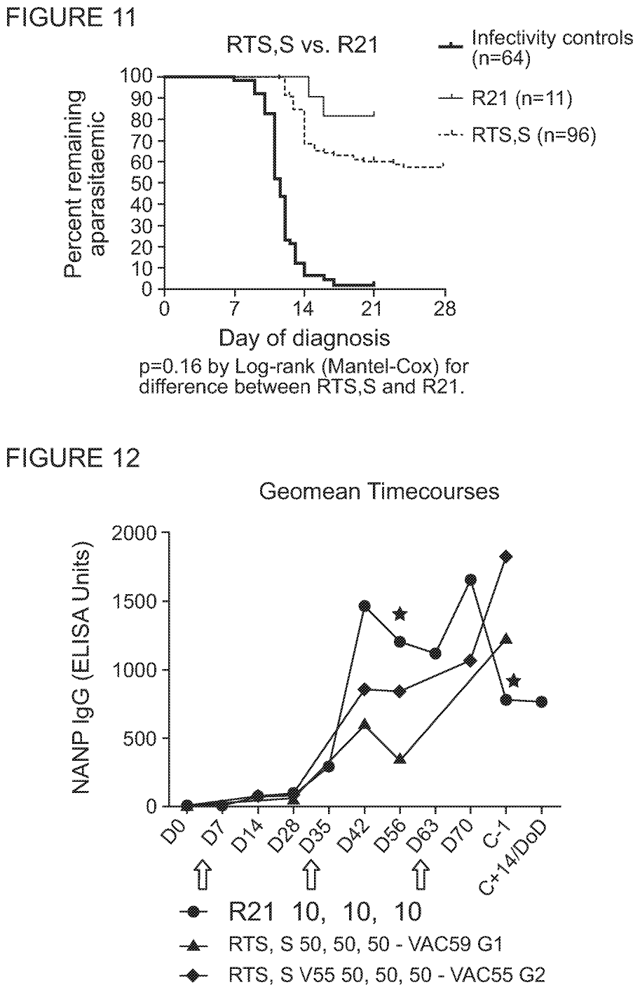

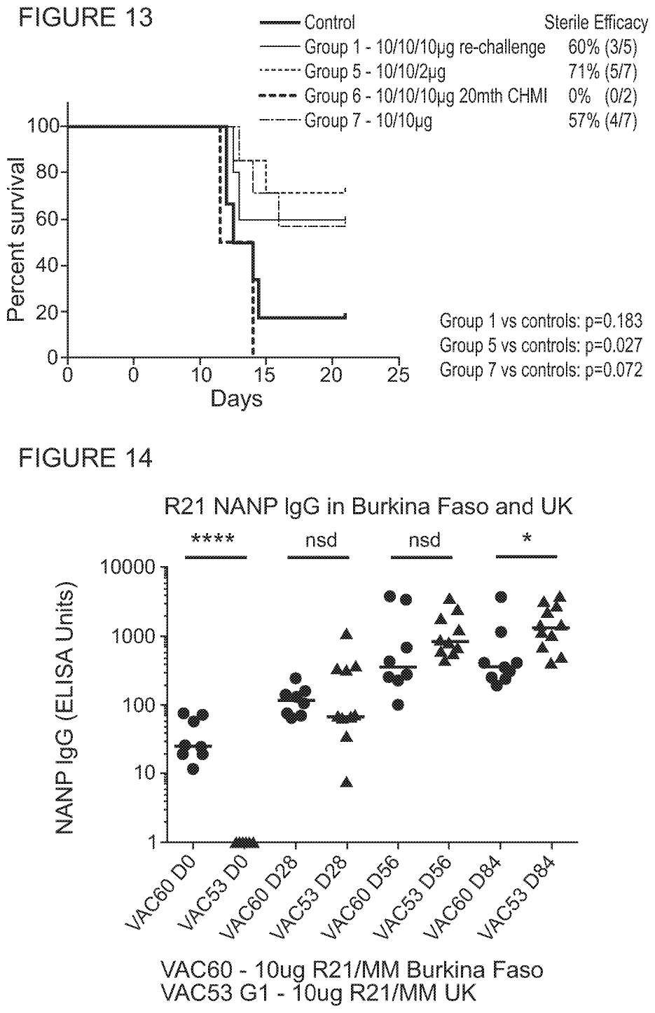

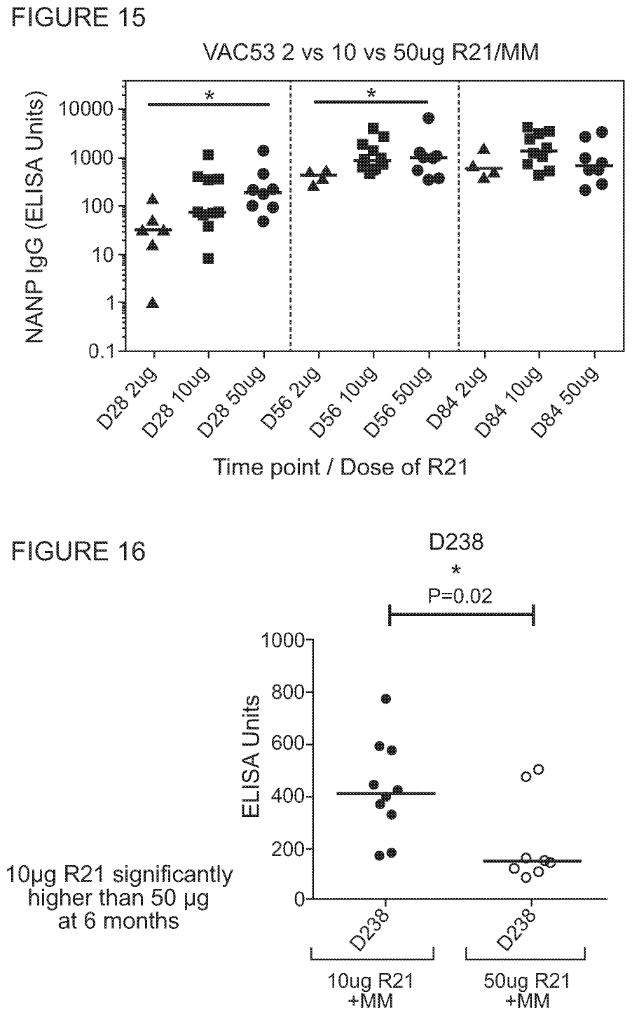

FIG. 7A: Percentage of subsets within total cTfh. G1 (10,10,10 μg R21/MM) has a significantly higher proportion of Tfh2 cTfh than G2 (50,50,10 μg R21/MM, Mann-Whitney analysis, P=0.0018). This higher Tfh2 response with the lower dose of R21/MM may account for the better performance of the lower dose of vaccine.

Boxed section (with arrow) indicates CXCR3-CCR6-cTfh are better at providing help to B cells (Locci et al Immunity 2013)

FIG. 7B: Percentage of CD19+CD20+ B cells within lymphocytes. Significantly higher percentage of CD19+CD20+ B cells within lymphocytes in the 10,10,10 μg R21/MM group than 50,50,10 μg R21/MM group (Mann-Whitney analysis, P=0.0104).

FIG. 7C: Percentage of switched memory B cells (CD19+CD20+IgD-CD27+) within lymphocytes. There is a significantly higher percentage of switched memory B cells within lymphocytes in 10,10,10 μg R21/MM group (Mann-Whitney analysis, P=0.0004), which again may relate to the better performance of the lower dose of vaccine.

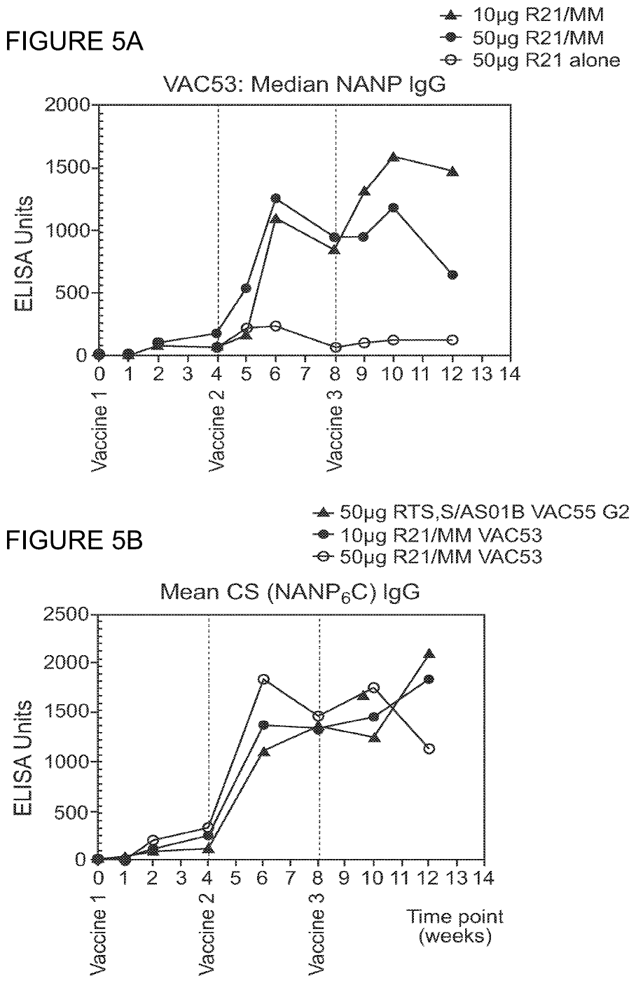

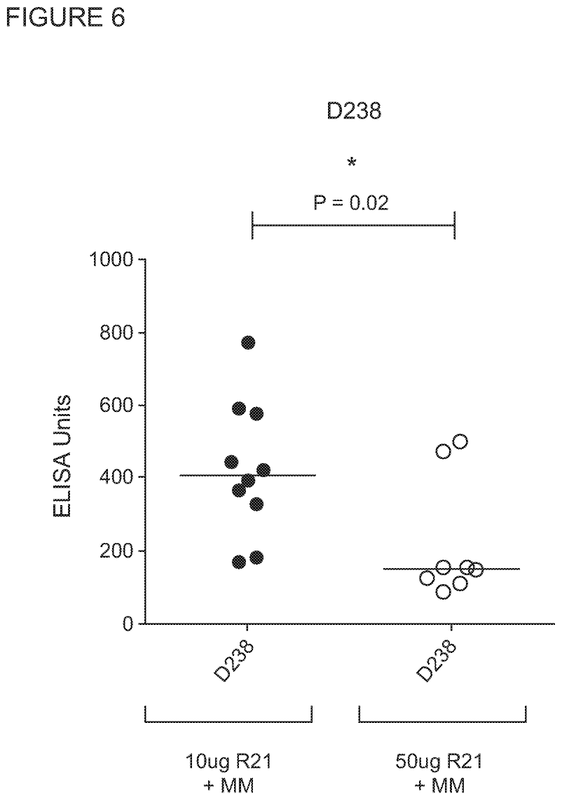

FIG. 8 shows plots. Very Low 2 μg Dose R21/Matrix-M; Still high immunogenicity with this very low dose; Reactogenicity was found to be minimal at this very low dose. NANP-specific IgG responses measured by ELISA as in FIGS. 5A, 5B, and 6. Comparison of NANP-specific antibody responses at day 28 (D28), D56 and D84 in VAC53 in volunteers vaccinated with 2, 10 or 50 μg R21 in MM. Assay was completed for all samples that were available at the time of testing-some volunteers had not yet passed these time points. Kruskal-Wallis analysis with Dunn's test for multiple comparisons at each time point shows a significantly lower titre in the 2 μg group at D28 and D56 but not at day 84 (Kruskal-Wallis P=0.022, P=0.050 and P=0.212 at D28, D56 and D84 respectively). Lines indicated medians in each group at each time point. The similar titres at day 84 across the dose groups suggests that even this very low dose of R21 may well be protective.

FIG. 9 shows a table. VAC065-Phase I/IIa Sporozoite Challenge Study; 31 vaccinees (11, 11, 9 in Groups 1, 2 and 3) & 6 controls underwent CHMI on 30 and 31 Jan. 2017. Summary of the design and groups within Vac065, the phase IIa controlled human malaria infection (CHMI) trial conducted in Oxford in 2016-2017.

FIG. 10 shows a graph. 82% Efficacy with Low Dose R21/Matrix-M.

| | Proportion | |
|---|---|---|
| | protected (%) | P value |
| Group 1 | 9/11 (82%) | P = 0.0009 |
| 10, 10, 10 | | |
| Group 2 | 7/11 (64%) | P = 0.004 |
| 50, 50, 10 | | |
| Group 3 | | |
| 10, 10, 10 + | 6/9 (67%) | P = 0.006 |
| vectors | | |

Summary of the efficacy of the thee vaccine regimens in Groups 1-3 in the Vac065 CHMI trial. The highest efficacy was observed, surprisingly, with the simple low dose regimen of group 1, in which three 4 weekly doses of 10 micrograms of R21 in 50 micrograms of matrix-M was used with a vaccine to adjuvant ratio of 1:5. Lower efficacy was observed in the other groups. The Kaplan-Meier curve of time to malaria diagnosis shows that in Group 1 the two (of eleven) vaccinees who did develop malaria did so several days later then in the control subjects. This indicates that the vaccine has reduced the number of parasites leading to a blood-stage infection so that parasites are detected later. Hence, in these two subjects there was clear evidence of partial vaccine efficacy.

FIG. 11 shows a graph. Efficacy of Three Doses of 10 μg of R21/Matrix-M vs 50 μg of RTS,S/AS01.

Comparison of the efficacy of the R21 in matrix-M vaccine to the efficacy of the RTS,S/AS01 vaccine, used in a standard 0, 4, 8 week regime with 50 mcg of RTS,S in AS01, in previous CHMI trials in the UK and the USA, compared to non-vaccinated controls in the same trials. The efficacy of the R21 vaccine is higher at 82% than that of the RTS,S vaccine which is 58% and this difference approaches statistical significance (two-tailed P value=0.16; one-tailed P value=0.08).

FIG. 12 shows a graph. Immunogenicity after Two Doses of R21/Matrix-M May Be Higher than after Three (Day, 0, 28, 56 regime).

Geometric mean NANP IgG timecourses (measured using the same ELISA method as previous figures). Time courses are shown for 10 μg R21/MM (VAC53 G1) and 50 μg RTS,S/AS01B in VAC55 G2 and VAC59 G1. Antibody responses after the second vaccination are significantly higher in the 10 μg R21/MM group compared to either of the 50 μg RTS,S/AS01B groups. Although these titres are re-boosted by a third vaccination, they are boost to levels comparable to the peak post-second dose and drop more rapidly than after the second vaccination. This high level immunogenicity after just two doses, comparable in titre to the levels observed after three doses, suggests strongly that, unexpectedly, a two dose regime of R21 may provide significant efficacy (as does the three dose regimen).

FIG. 13 shows graphs.

FIG. 14 shows a plot.

FIG. 15 shows a plot.

FIG. 16 shows a plot.

### EXAMPLES

### Example 1: A Safety and Efficacy Study of R21+/−ChAd63/MVA ME-TRAP

### Sponsor:

University of Oxford

### Information Provided by (Responsible Party):

University of Oxford

### ClinicalTrials.gov Identifier:

NCT02905019

### Purpose

The purpose of this study is to assess the safety and efficacy of adjuvanted R21 alone and in combination with a viral-vectored vaccine regimen (constituting adjuvanted R21+ChAd63 and MVA encoding ME-TRAP) against malaria sporozoite challenge in healthy malaria-naive volunteers.

Healthy adult volunteers will be recruited in London, Oxford and Southampton.

All vaccinations will be administered intramuscularly. The study involves having either two, three or five vaccinations and then undergoing challenge infection with malaria, or receiving no vaccinations then undergoing challenge infection with malaria.

| Condition | Intervention | Phase |
|---|---|---|
| Malaria | Biological: R21 with Matrix-M1 | Phase 1 |
| | Biological: ChAd63 ME-TRAP | Phase 2 |
| | Biological: MVA ME-TRAP | |

Study Type: Interventional
Study Design: Allocation: Randomized
   Intervention Model: Parallel Assignment
   Masking: Outcomes Assessor
   Primary Purpose: Prevention
Official Title: A Phase I/IIa Sporozoite Challenge Study to Assess the Safety and Protective Efficacy of Adjuvanted R21 at Two Different Doses and the Combination Malaria Vaccine Candidate Regimen of Adjuvanted R21+ChAd63 and MVA Encoding ME-TRAP.

### Resource Links Provided by NLM:

MedlinePlus related topics: Malaria
Genetic and Rare Diseases Information Center resources: Malaria
U.S. FDA Resources

### Further Study Details as Provided by University of Oxford:

Primary Outcome Measures:
   Efficacy of adjuvanted R21 at two different doses and adjuvanted R21+ChAd63 and MVA encoding ME-TRAP in healthy malaria-naïve volunteers as assessed by number of completely protected individuals. [Time Frame: 6 months]
Use statistical analysis to compare number of completely protected individuals (those who do not, by Day 21 following sporozoite challenge, develop blood stage infection measured by occurrence of P. falciparum parasitemia, assessed by blood slide) in the vaccine groups compared to the controls.
   Safety of adjuvanted R21 at two different doses and adjuvanted R21+ChAd63 and MVA encoding ME-TRAP in healthy malaria-naïve volunteers as assessed by frequency of adverse events. [Time Frame: 6 months]
Solicited and unsolicited adverse event data will be collected at each clinic visit from diary cards, clinical review, clinical examination (including observations) and laboratory results. This AE data will be tabulated and frequency, duration and severity of AEs compared between groups.

### Secondary Outcome Measures:

Humoral immunogenicity generated in malaria naïve individuals with adjuvanted R21 at two different doses [Time Frame: 6 months]

Antibody response to the circumsporozoite protein generated by vaccination with adjuvanted R21.
Cell-mediated immunogenicity generated in malaria naïve individuals with ChAd63 and MVA encoding ME-TRAP [Time Frame: 6 months]

T-cell responses to the TRAP antigen of the malaria parasite generated by vaccination with ChAd63 and MVA encoding ME-TRAP.
Efficacy measured as time to P. falciparum parasitemia assessed by PCR against malaria sporozoite challenge, in healthy malaria-naïve volunteers. [Time Frame: 6 months]

Statistical analyses using blood stage infection as defined by 500 or more parasites/ml in peripheral blood by quantitative PCR.
Efficacy measured as time to P. falciparum parasitemia assessed by blood slide against malaria sporozoite challenge, in healthy malaria-naïve volunteers. [Time Frame: 6 months]

Statistical analyses using blood stage infection defined by a composite of symptoms, blood film result and parasitaemia.
Efficacy measured as time to P. falciparum parasitemia assessed by parasite density dynamics assessed by PCR against malaria sporozoite challenge, in healthy malaria-naïve volunteers. [Time Frame: 6 months]

Statistical analyses using blood stage malaria infection as defined by 20 or more P. falciparum parasites/ml in peripheral blood by quantitative PCR.

### Other Outcome Measures:

Long term protective efficacy of adjuvanted R21 at two different doses and adjuvanted R21+ChAd63 and MVA encoding ME-TRAP [Time Frame: 12 months]

Long term efficacy of the vaccination regimens will be assessed by re-challenging any sterilely protected individuals at 5-7 months after the first sporozoite challenge (˜12 months after the start of the study) and comparing the number of re-challenges who develop blood stage infection with unvaccinated controls.

Estimated Enrollment: 70

| | Assigned |
|---|---|
| Arms | Interventions |
| Active Comparator: Group 1 | Biological: R21 |
| R21 with Matrix-M1. Three vaccinations with 10 | with Matrix-M1 |
| μg R21/50 μg Matrix-M1 on days 0, 28 and 56. | Vaccine |
| Active Comparator: Group 2 | Biological: R21 |
| R21 with Matrix-M1. Two vaccinations with 50 μg | with Matrix-M1 |
| R21/50 μg Matrix-M1 on days 0 and 28 and one | Vaccine |
| vaccination with 10 μg R21/50 μg Matrix M1 on | |
| day 56. | |
| Active Comparator: Group 3 | Biological: R21 |
| R21 with Matrix-M1, ChAd63 ME-TRAP and | with Matrix-M1 |
| MVA ME-TRAP. Three vaccinations with 10 μg | Vaccine |
| R21/50 μg Matrix-M1 on days 0, 28 and 56. Plus | Biological: ChAd63 |
| one vaccination with ChAd63 ME-TRAP on day 7 | ME-TRAP |
| and one vaccination with MVA ME-TRAP | Vaccine |
| on day 63. | Biological: MVA |
| | ME-TRAP |
| | Vaccine |
| No Intervention: Group 4a | |
| These volunteers will not be vaccinated and will | |
| serve as infectivity controls when groups | |
| 1-3 undergo challenge. | |
| No Intervention: Group 4b | |
| These volunteers will not be vaccinated and will | |
| serve as infectivity controls when group 5-7 | |
| and sterilely protected volunteers from groups | |
| 1-3 undergo challenge. | |
| No Intervention: Group 4c | |
| These volunteers will not be vaccinated and will | |
| serve as infectivity controls if any volunteers | |
| from groups 5 and 7 are rechallenged. | |
| Active Comparator: Group 5 | Biological: R21 |
| R21 with Matrix-M1. Two vaccinations with | with Matrix-M1 |
| 10 μg R21/50 μg Matrix-M1 on days 0 and | Vaccine |
| 28 and one vaccination with 2 μg R21/50 μg | |
| Matrix-M1 on day 56. | |
| No Intervention: Group 6 | |
| Volunteers in this group have received vaccinations | |
| in a different malaria vaccine trial. These volunteers | |
| will not receive any vaccinations in this trial, but | |
| will undergo controlled human malaria infection | |
| as part of this study. | |
| Active Comparator: Group 7 | Biological: R21 |
| R21 with Matrix-M1. Two vaccinations with | with Matrix-M1 |
| 10 μg R21/50 μg Matrix-M1 on days 0 and 28. | Vaccine |

### DETAILED DESCRIPTION

Vaccination phases and challenge procedures have been staggered over the trial period into 2 parts, challenge A and B.

### Challenge A:

Groups 1-3 consist of volunteers receiving either R21 alone or R21+ChAd63-MVA ME-TRAP followed by CHMI by sporozoite challenge (mosquito bite) at week 12. Twelve volunteers will be recruited to each group.
Group 4a will serve as infectivity controls, these volunteers will not be vaccinated.

### Challenge B:

Sterilely protected volunteers in groups 1-3 may be rechallenged to assess durability of efficacy, 5-12 months after the initial challenge.
Groups 5-7 will also be enrolled to participate in challenge B.
Group 5 (8 volunteers) will test the efficacy of standard dose R21 with a fractional third dose followed by CHMI at week 12.
Group 6 will test the long-term efficacy of the standard dose R21 vaccination regimen (volunteers in this group will have already received their vaccinations whilst enrolled in the VAC053 phase I malaria trial which started in 2015 and will therefore not receive any additional vaccinations before undergoing challenge approximately two years after their immunisations).
Group 7 (8 volunteers) will test the efficacy of a two dose R21 vaccination regimen followed by CHMI at week 8.
Group 4b will serve as infectivity controls for groups 5-7 and sterilely protected group 1-3 volunteers. Group 4c volunteers will be used as infectivity controls if any volunteers from groups 5 and 7 are rechallenged.

### Eligibility:

Ages Eligible for Study: 18 Years to 45 Years (Adult)
Sexes Eligible for Study: All
Accepts Healthy Volunteers: Yes

### Criteria

### Inclusion Criteria:

Healthy adults aged 18 to 45 years.
Able and willing (in the Investigator's opinion) to comply with all study requirements.
Willing to allow the investigators to discuss the volunteer's medical history with their General Practitioner.
Women only: Must practice continuous effective contraception* for the duration of the study.
Agreement to refrain from blood donation during the course of the study and for at least 3 years after the end of their involvement in the study.
Written informed consent to participate in the trial.
Reachable (24/7) by mobile phone during the period between CHMI and completion of antimalarial treatment.
Willingness to take a curative anti-malaria regimen following CHMI.
For volunteers not living close to their designated malaria challenge follow-up site (Oxford or Southampton): agreement to stay in a hotel room close to the trial centre during a part of the study (from at least day 6.5 post mosquito bite until anti-malarial treatment is completed).
Answer all questions on the informed consent quiz correctly.

### Exclusion Criteria:

History of clinical malaria (any species).
Travel to a clearly malaria endemic locality during the study period or within the preceding six months
Use of systemic antibiotics with known antimalarial activity within 30 days of CHMI (e.g. trimethoprim-sulfamethoxazole, doxycycline, tetracycline, clindamycin, erythromycin, fluoroquinolones and azithromycin)
Receipt of an investigational product in the 30 days preceding enrolment, or planned receipt during the study period.
Prior receipt of an investigational vaccine likely to impact on interpretation of the trial data as assessed by the investigator. If any volunteers in Group 1-3 undergo rechallenge, this exclusion criterion does not extend to the vaccines previously received in the VAC065 trial.
For Group 3 volunteers only: prior receipt of a non-malaria MVA or non-malaria adenovirus vectored experimental vaccine
Any confirmed or suspected immunosuppressive or immunodeficient state, including HIV infection; asplenia; recurrent, severe infections and chronic (more than 14 days) immunosuppressant medication within the past 6 months (inhaled and topical steroids are allowed).
Use of immunoglobulins or blood products within 3 months prior to enrolment.
History of allergic disease or reactions likely to be exacerbated by any component of the vaccine (e.g. egg products, Kathon) or malaria infection.
Any history of anaphylaxis post vaccination.
History of clinically significant contact dermatitis.
History of sickle cell anaemia, sickle cell trait, thalassaemia or thalassaemia trait or any haematological condition that could affect susceptibility to malaria infection.
Pregnancy, lactation or intention to become pregnant during the study.
Use of medications known to cause prolongation of the QT interval and existing contraindication to the use of Malarone™
Use of medications known to have a potentially clinically significant interaction with Riamet™ and Malarone™
Any clinical condition known to prolong the QT interval
History of cardiac arrhythmia, including clinically relevant bradycardia
Disturbances of electrolyte balance, eg, hypokalemia or hypomagnesaemia
Family history of congenital QT prolongation or sudden death.
Contraindications to the use of all three proposed anti-malarial medications; Riamet™ Malarone™ and Chloroquine.
History of cancer (except basal cell carcinoma of the skin and cervical carcinoma in situ).
History of serious psychiatric condition that may affect participation in the study.
Any other serious chronic illness requiring hospital specialist supervision.
Suspected or known current alcohol abuse as defined by an alcohol intake of greater than 42 standard UK units every week.
Suspected or known injecting drug abuse in the 5 years preceding enrolment.
Hepatitis B surface antigen (HBsAg) detected in serum.
Seropositive for hepatitis C virus (antibodies to HCV) at screening (unless has taken part in a prior hepatitis C vaccine study with confirmed negative HCV antibodies prior to participation in that study, and negative HCV RNA PCR at screening for this study).
An estimated, ten year risk of fatal cardiovascular disease of ≥5%, as estimated by the Systematic Coronary Risk Evaluation (SCORE) system.60
Positive family history in 1st and 2nd degree relatives<50 years old for cardiac disease.
Volunteers unable to be closely followed for social, geographic or psychological reasons.
Any clinically significant abnormal finding on biochemistry or haematology blood tests, urinalysis or clinical examination.
Any other significant disease, disorder, or finding which may significantly increase the risk to the volunteer because of participation in the study, affect the ability of the volunteer to participate in the study or impair interpretation of the study data.

ClinicalTrials.gov identifier: NCT02905019

### Locations-United Kingdom

NIHR Wellcome Trust Clinical Research Facility, Hammersmith Hospital
   London, United Kingdom
   Contact: Reshma Sultan +44 (0) 20 331 31086
CCV™, University of Oxford,
   Oxford, United Kingdom, OX3 7LE
   Contact: Volunteer Coordinator vaccinetrials@ndm.ox.ac.uk
Southampton National Institute for Health Research
   Southampton, United Kingdom
   Contact 02381 204989 UHS.RecruitmentCRF@nhs.net

### Sponsors and Collaborators: University of Oxford

Responsible Party: University of Oxford
ClinicalTrials.gov Identifier: NCT02905019
Other Study ID Numbers: VAC065
Individual Participant Data (IPD) Sharing Statement:
Plan to Share IPD: Undecided

### Example 2

With reference to the clinical trial outline, there was good reason for believing that either groups 2 or 3 would have been better and surprisingly they were not. It is impressive that the Group 1 result (82% efficacy) with low dose R21 was better than the other two regimens tested (Groups 2 and 3). Group 2 might have been better because in it a larger amount of R21 was administered: 50 mcg rather than 10 mcg for doses 1 and 2, but the same dose for dose 3: there is evidence in the literature from RTS,S that such a “fractional (i.e. reduced) third dose” regime might be better than a standard regime (as in group 1) (reference Regules et al. J Infect Dis. 2016; 214:762-71.) Group 3 might have been the best group because of the additional administration of partially effective vectored vaccines, as reported by Rampling et al. (J Infect Dis. 2016 Sep. 1; 214 (5): 772-81), but strikingly the efficacy in Group 3 was not as high as in Group 1.

### Example 3

Overview: R21 is a novel malaria vaccine candidate, which is a biosimilar of the most advanced malaria vaccine candidate, RTS,S/AS01 and is composed of a fusion protein of the malaria circumsporozoite protein and Hepatitis B surface antigen. We assessed the efficacy of R21 administered with Matrix-M (R21/MM) given alone at two different dose schedules and in combination with viral-vectored vaccines using controlled human malaria infection (CHMI) in healthy UK volunteers.

We undertook this Phase IIa study in healthy UK volunteers to assess the efficacy against malaria sporozoite challenge of R21/MM in different dose schedules and in combination with ChAd63-MVA ME-TRAP.

Methods Volunteers were recruited into this Phase IIa study at three trial centres in the UK and CHMI was undertaken at Imperial College, London. Thirty-one healthy volunteers were vaccinated with either 3 doses of 10/10/10 μg of R21/MM (Group 1; n=11), or 3 doses of 50/50/10 μg of R21/MM (Group 2; n=11), or 3 doses of 10/10/10 μg of R21/MM (Group 1; n=11) given with ChAd63-MVA expressing ME-TRAP.

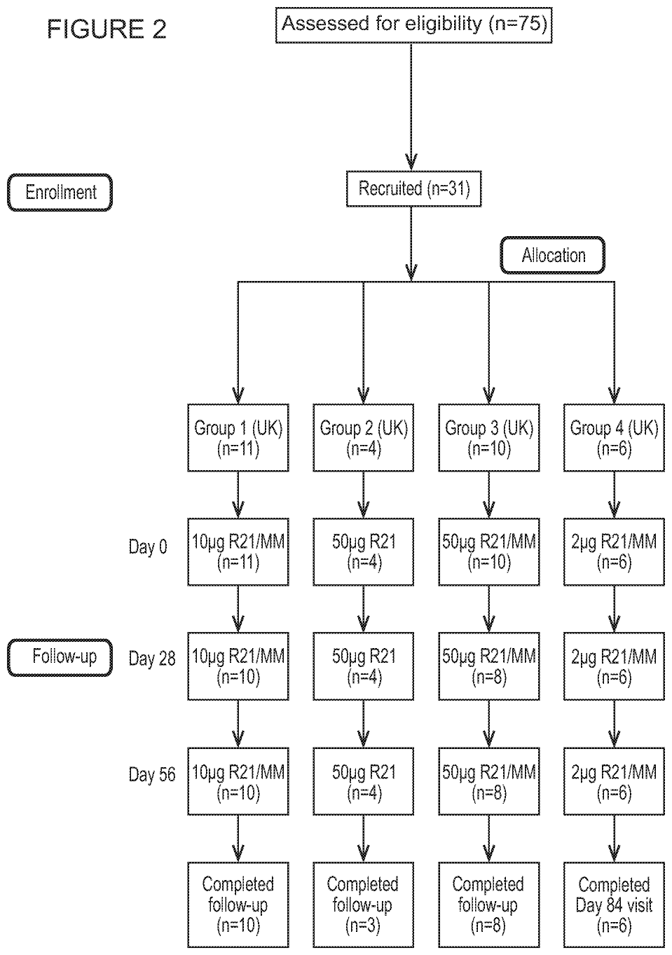

Referring to FIG. 2, we described a Phase Ia study; Open-label; Non-randomised; Healthy adults aged 18 to 50 years; Oxford; London (Imperial); 31 volunteers in total.

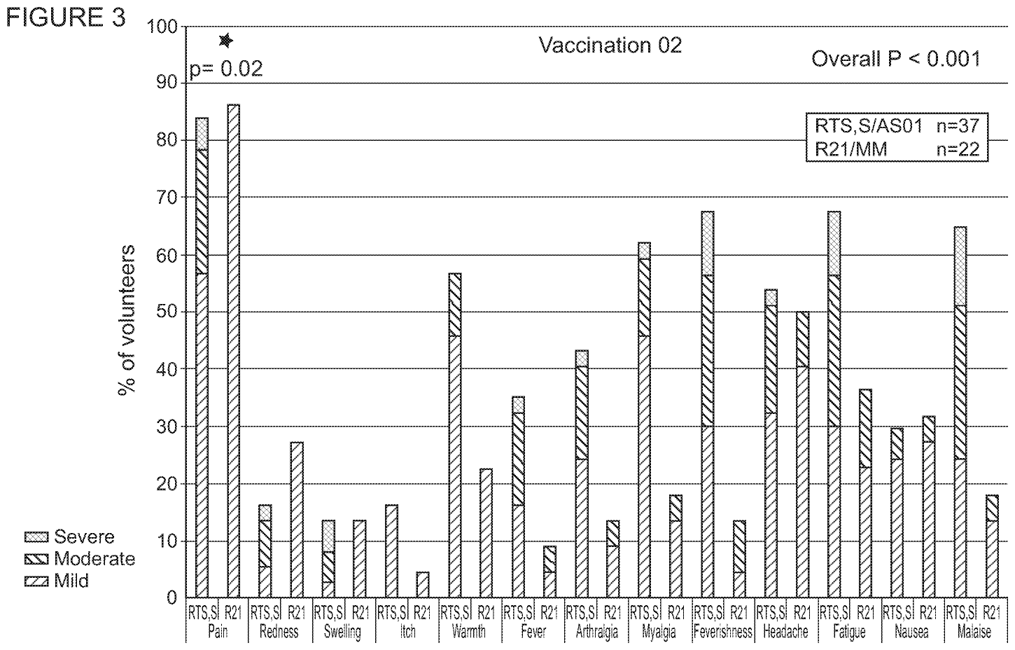

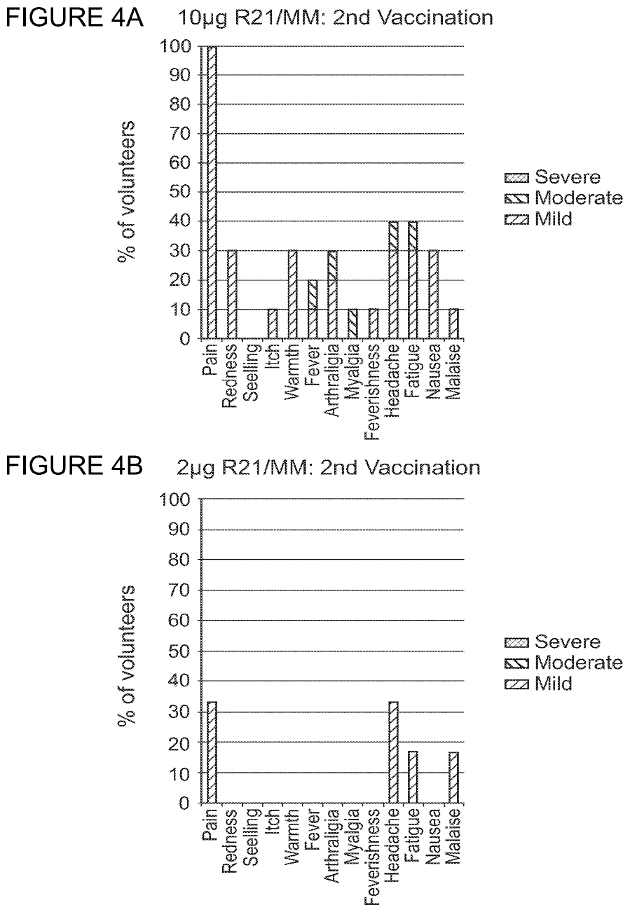

As observed in the phase I trial (Vac053, see FIG. 2 for the trial profile) the safety profile of R21 (10 micrograms) in matrix-M (50 micrograms), see FIG. 3, was clearly better than for the RTS,S/AS01 vaccine. As in the phase I trial, vac053—see FIGS. 5A and 5B, good immunogenicity was observed for antibodies to CSP with all regimens.

CHMI was delivered by mosquito bite at week 12 after first vaccination, including 6 unvaccinated controls. The trial is registered with ClinicalTrials.gov (NCT02905019)

Findings This trial was done between 7 Nov. 2016 and 15 May 2017. Of over 70 volunteers screened, 37 volunteers (FIG. 9) underwent malaria sporozoite challenge on the 30 and 31 Jan. 2017. Vaccinations were generally well tolerated, with the majority of local and systemic adverse events being mild in nature and an improved safety profile compared to published RTS,S/As01 data-see FIG. 3. Sterile protection was observed (see FIG. 10) in 9/11 (81.8%) subjects in Group 1, 7/11 (63.6%) subjects in Group 2 and 6/9 (66.7%) subjects in Group 3. All vaccinated volunteers showed a significant delay in patency in comparison to control volunteers. 5/6 control subjects were diagnosed with blood stage malaria. Antibody responses to the NANP repeat region of the circumsporozoite protein were significantly boosted at 14 days after the 2nd vaccination in all volunteers and comparable to RTS,S/AS01.

High level efficacy observed in the lower dose group is demonstrated as a benefit of the invention.

### Experimental Details

### Study Design and Participants

We did a Phase IIa study in healthy malaria-naïve adult males and non-pregnant females between the ages of 18 and 45 years. Recruitment and vaccination were conducted at the Centre for Clinical Vaccinology and Tropical Medicine at the University of Oxford and the Wellcome Trust Clinical Research Facility in Southampton and Imperial College in the United Kingdom. This Phase IIa, open-label malaria sporozoite challenge trial consisted of 4 cohorts. The sample sizes reflect practical limitations on volunteer recruitment, ethical considerations limiting the number of volunteers that should receive a vaccine regimen without prior evidence of efficacy, and the desire to describe the efficacy of the immunisation regimes. Allocation to study group was undertaken by the investigators prior to enrolment based on subject preference. Group 1 (n=11) received 3 vaccinations (R21/MM 10 μg at 0, 4 and 8 weeks); Group 2 (n=11) received 3 vaccinations (R21/MM 50 μg at 0 and 4 weeks and R21/MM 10 μg at 8 weeks); Group 3 (n=9) received 5 vaccinations (R21/MM 10 μg at 0, 4 and 8 weeks and ChAd63 ME-TRAP 5×10¹⁰ virus particles (vp) at 1 week, and MVA ME-TRAP 2×10⁸ plaque forming units (pfu) at 9 weeks) and Group 4 (n=6) received no vaccinations. All subjects underwent initial CHMI by mosquito bite at the same time (week 12 after first vaccination for vaccinated subjects).

The volunteers were infected using five infectious bites from P. falciparum 3D7-strain infected Anopheles stephensi mosquitoes at Imperial College, London. All subjects were infected with a single batch of mosquitoes supplied by the Department of Entomology, Walter Reed Army Institute of Research, Washington DC, USA. The inclusion and exclusion criteria are listed in the supplementary appendix. All participants gave written informed consent prior to participation, and the study was conducted according to the principles of the Declaration of Helsinki and in accordance with Good Clinical Practice (GCP).

The study was approved by the UK National Research Ethics Service, Committee South Central-Berkshire (Ref: 16/SC/0261), the Medicines and Healthcare Products Regulatory Agency (Ref: 21584/0360/001-0001), and the Oxford University Clinical Trials and Research Governance team, who independently and externally monitored compliance with Good Clinical Practice guidelines. Viral-vectored vaccine use was authorised by the Genetically Modified Organisms Safety Committee (GMSC) of the Oxford University Hospitals NHS Trust (Reference number GM 462.16.88). The trial was registered with ClinicalTrials.gov (Ref: NCT02905019) and an independent local safety monitor provided safety oversight.

### Procedures

R21 (Batch no: 01015-01) was manufactured and vialed under Good Manufacturing Practice conditions at the Clinical Biomanufacturing Facility, University of Oxford: the production, manufacture and storage of this product have been previously described in in WO2014/111733; see also Venkatraman et al. Matrix-M (Batch no: M1-103) is a patented adjuvant technology developed by Novavax: the production, manufacture and storage of this product have been previously described [21]. Generation, manufacture and storage of the ChAd63 ME-TRAP (Batch no: 01S11-01) and MVA ME-TRAP (Batch no: 0091013) vaccines has been previously described [12, 22]. On the day of vaccination, R21 was thawed to room temperature and was administered intramuscularly into the deltoid of the non-dominant arm within 1 hour of removal from the freezer, mixed with Matrix-M. The viral vectored vaccines were administered intramuscularly within 1 hour of thawing into the deltoid of the dominant arm (the contralateral arm to R21 administration). All volunteers were observed in the unit for 1 hour after vaccination. Volunteers were provided with an electronic diary card to record their temperature and any solicited local and systemic adverse events for 7 days post-vaccination and unsolicited adverse events for 28 days post-vaccination. Severity grading of adverse events and the assignment of a causal relationship for adverse events were conducted according to predefined guidelines stated in the protocol. An independent Safety Monitoring Committee provided safety oversight during the course of the trial. Safety bloods including full blood count, renal function and liver function tests were done on visits at day 0, 7, 28, 35, 56, 63 and 83 (day before CHMI) in Group 1 and 2 volunteers. Additional safety bloods were done on day 14 and 70 for volunteers in Group 3. Antibody responses measured by anti-NANP IgG ELISA were performed on samples from days 0, 7, 14, 28, 35, 42, 56, 63, 70 and 83. Ex-vivo IFN-ELISpot responses to CSP were assessed on samples from day 0, 42 and 83. In addition, IFN-ELISpot responses to TRAP were assessed on samples from day 0, 28, 70 and 83 in Group 3 volunteers. The full methods used for these immunological assays have been previously described [23]. The CHMI procedure was as described by Rampling et al. J Infect Dis. 2016 Sep. 1; 214 (5): 772-81. Following CHMI, a diagnosis of blood stage malaria infection was made in subjects with symptoms suggestive of malaria and positive thick film microscopy, or qPCR result>500 parasites/ml if either thick film was negative, or symptoms were absent [12]. Vaccinated subjects who had not developed blood stage malaria by day 21 after CHMI were deemed to exhibit sterile protection.

### Outcomes

The primary outcome measures were to assess the efficacy (occurrence of P. falciparum parasitemia, assessed by blood slide) of the different vaccine regimens against malaria sporozoite challenge, and to assess the safety of the vaccines, in healthy malaria-naïve volunteers. The secondary outcome measures were to assess immunogenicity and to assess the efficacy (measured as time to P. falciparum parasitemia assessed by blood slide, by PCR, and parasite density dynamics assessed by PCR) in healthy malaria-naïve volunteers.

### Statistical Analysis

Data were analyzed using GraphPad Prism version 5.03 for Windows (GraphPad Software Inc., California, USA) and Stata 10.0 (Statacorp LP, Texas, USA). Geometric means or medians with interquartile ranges for each group are described. Kruskal-Wallis analysis and the Friedman test were used to compare peak immune responses with the baseline. Significance testing of differences between two groups used Mann-Whitney analysis. A Wilcoxon matched-pairs analysis was used to compare between time points within groups. A chi-squared test for trend was used to compare the safety data between different groups. A statistically significant difference in efficacy between a vaccination regimen and controls was assessed by log rank analysis of Kaplan Meier curves, using a one-tailed log rank test at the different endpoints. A value of p<0.05 was considered significant.

### Results

### Study Population

From 7 Nov. 2016 to 31 Jan. 2017, a total of 43 volunteers of the 75 who were screened for eligibility were enrolled into this study. One volunteer in Group 1 withdrew after their first vaccination due to a change in personal circumstances and was replaced. Another Group 1 volunteer withdrew after their second vaccination due to a change in personal circumstances. One Group 2 volunteer withdrew after their first vaccination as they were no longer able to commit to the schedule of attendances. Two volunteers in Group 3 withdrew after their third vaccination due to a change in personal circumstances. Another Group 3 volunteer withdrew after their fifth vaccination due to apprehensions about undergoing CHMI. None of the withdrawals were related to the vaccination and there were no ongoing AEs and safety bloods were normal. 37 volunteers (11 Group 1 volunteers, 11 Group 2 volunteers, 9 Group 3 volunteers and 6 unvaccinated controls) underwent CHMI on the 30 and 31 Jan. 2017. All of these 37 volunteers completed follow-up until 90 days post-CHMI. Participant flow and study design is summarised in the table of FIG. 9.

### Efficacy

There were 9/11 Group 1 volunteers, 7/11 Group 2 volunteers and 6/9 Group 3 volunteers who hadn't developed malaria at 21 days post-CHMI resulting in a sterile efficacy of 81.8% (p=0.0009), 63.6% (p=0.004) and 66.7% (p=0.006), respectively. Five out of six control volunteers developed malaria with a mean time to diagnosis of 11.3 days (Median 11, range 11-12, SD 0.45). A significant delay in patency was observed in all three groups with a mean time to diagnosis of 15.3 days (Median 15.3, range 14.5-16, SD 1.1), 16.4 days (Median 16.5, range 14.5-16, SD 1.5) and 15.3 days (Median 16, range 14-16, SD 1.2) in Groups 1, 2 and 3 respectively.

### Adverse Events

No serious adverse reactions (SARs) or suspected unexpected serious adverse reactions (SUSARs) occurred. There were no withdrawals due to safety concerns and no pre-defined study stopping or holding rules were activated. The majority of adverse events (AEs) reported were self-limiting and mild in severity and reactogenicity profiles observed were similar to previous studies for both the R21/MM (Ref: VAC053, FIG. 2; Venkatraman et al) and the ChAd63_MVA ME-TRAP [23]. Solicited local and systemic adverse events in the first 7 days after each vaccination were mild to moderate and overall all vaccination regimes were well tolerated. Vaccine site pain was the most common local adverse event and was predominantly mild in severity. The reactogenicity profile of the 10/10/10 μg R21/MM dose group, which has also been tested in a previous Phase I trial (Venkatraman et al) was significantly improved in comparison to data from two previous trials in our centre using RTS,S/AS01 (Vaccination 1—p=0.003; Vaccination 2—p=0; Vaccination 3—p=0.125). Vaccine site pain was significantly reduced after the first (p=0.02) and second (p=0.02) vaccinations in comparison to RTS,S/AS01. Unsolicited adverse events collected for 28 days after each vaccination deemed definitely, possibly or probably related to vaccination were predominantly mild in nature. Laboratory adverse events were predominantly Grade 1. Immunogenicity profiling showed potent antibody induction to the central NANP repeat region of the circumsporozoite protein as observed in the Vac053 phase I trial (FIGS. 5A and 5B).

### Discussion

We report here for the first time, high level efficacy of a novel malaria vaccine candidate, R21 adjuvanted with Matrix-M at one-fifth of the dose of the most advanced malaria vaccine candidate, RTS,S/AS01. This will have significant dose-sparing and cost-saving advantages for large scale production of the vaccine if it was proven to be efficacious in subjects living in malaria endemic regions. Our data confirms that this vaccine approach is safe and well tolerated in healthy UK volunteers with adverse events being predominantly mild in nature. The use of a 50 μg dose with a fractional (⅕^{th}) third dose did not improve efficacy, in contrast to the recently published study by Regules et al., where it was shown that a delayed fractional third dose of RTS,S/AS01 improved efficacy in a malaria sporozoite challenge study [10]. The humoral response to the vaccine did not vary between Groups 1-3 and the addition of the viral-vectored vaccines to the regime in Group 3 did not result in reduced CS antibody immunogenicity. The magnitude of the response did not predict efficacy, which suggests that the quality or other aspects of the antibody response may also be relevant for efficacy. There were minimal IFN-γ ELISpot responses to CSP induced by R21/MM, which is similar to previous experience with RTS,S/AS01 that only induces low level CD4⁺ and no CD8⁺ T-cell responses [24].

One of the control volunteers did not develop clinical malaria, which is very unusual and has never occurred previously in CHMI trials at the Oxford centre. However, the failure of one of the controls to develop malaria has been reported in a number of other CHMI trials undertaken by Sanaria Inc. to test the intravenous whole sporozoite vaccine [25, 26]. The mean time to patency in the control group of 11.3 days indicates that this was not an unusually weak challenge, and the vaccination and CHMI methodology used in this trial are largely comparable to other CHMI studies [23].

In conclusion, this Phase IIa malaria sporozoite challenge study demonstrated high level efficacy with a novel low-cost malaria vaccine, R21/MM. This high efficacy is at least as high or higher than regimes using a higher (50 microgram) dose of the RTS,S/AS01 vaccine used in a similar 0, 4, 8 week schedule (FIG. 11).

It could also be that a two-dose schedule would be sufficient to protect this primed population-based on the high immunogenicity observed for anti-CSP antibodies after just two immunisations (FIG. 12). In view of this, are using a two dose-schedule using 10 μg of R21/MM in a CHMI study in healthy UK volunteers. If this is proven to also have high level efficacy, this would have significant cost-saving benefits and would be potentially easier to deploy in co-ordination with the EPI program.

### Example 4—Challenge Study

Here we show a further challenge study of R21/matrix-M vaccines. We provide data from our completed challenge study with low-dose R21 in matrix-M. In summary we show efficacy in the following groups:—
Re-challenged volunteers at 8.5 months post-immunisation: 3/5 protected=60%
A two dose group (10, 10 mcgs): 4/7 protected at 3 weeks=57%
A three dose group (10, 10, 2 mcgs): 5/7 protected at three weeks=71%

The 57% with a two dose regimen is unprecedented in the whole malaria vaccine field, demonstrating the remarkable technical effects of the invention. The rechallenge efficacy of 60% at 8.5 months is excellent too.

### Study Details

A further challenge study of R21/matrix-M vaccines was undertaken. Two key objectives are described below.

The first objective was to evaluate the durability of protection of the 10 microgram (mcg) R21 dose in 50 mcg of matrix-M administered three times at four weekly intervals. In the original challenge study at the end of January 2017 nine of eleven vaccinees administered this regimen were sterilely protected. Of the nine protected subjects all were invited for a re-challenge in mid-September 2017 and 5 agreed to participate. Of the five re-challenges three were again sterilely protected and two were not, corresponding to 60% sterile efficacy. No booster vaccine dose was administered before the re-challenge so that the re-challenge of these subjects occurred about 8.5 months after their last vaccine dose at the start of January.

This evidence of durability of sterile protection in most protected subjects out to 8.5 months after last immunisation is very positive and compares favourably with the efficacy of RTS,S/AS01 protection which is generally measured at about 6 months post last dose. Efficacy of R21 at 8.5 month was 49% compared to the reported efficacy of RTS,S/AS vaccine at 5-8 months of 26% (see table).

The second objective was to assess two new R21 immunisation regimes using the preferred low dosages of R21 in matrix-M. In one group seven subjects were immunised with just two doses of 10 mcg of R21 in 50 mcg of matrix-M adjuvant. Challenge was at 3 to 4 weeks after the last dose. Of these seven individuals 4 were sterilely protected amounting to 57% vaccine efficacy, apparently the highest efficacy ever reported with a two dose malaria vaccine (from assessment by detailed literature review).

A further vaccination group used two doses of 10 mcg of R21/matrix-M at a four week interval followed by a 2 mcg R21 dose in 50 mcg matrix-M after a further four weeks. Seven subjects thus immunised were challenged 3-4 weeks later and 5 were sterilely protected, an efficacy rate of 71%. This is little different from the 9/11 protected with three doses of 10 mcg of R21 in 50 mcg of matrix-M. These data with three low doses of R21/matrix-M provide further evidence of its high level efficacy.

Exemplary data are provided below:

### R21 (Invention)

3×10 mcg doses at 0, 1, 2 months challenge at 4 weeks
9/11=82%
5 (of 9) re-challenged at month 8.5 post last dose
3/5=60%
   Overall R21 efficacy at 8.5 months post last dose=49%

### RTS,S

Regules et al. J Infect Dis 2016 3 week post last dose challenge
10/16=62.5%
5 of 10 re-challenged at 8 months post last dose
1/5=20%
   Overall efficacy at 8.5 months post last dose=12.5%
Kester et al. J Infect Dis 2009 0, 1, 2 months: challenge at 2-3 weeks:
18/36 protected=50%
Rechallenge at 5.5 months post last dose:
4/9 protected=44%
   Overall efficacy of RTS,S/AS01 at 5.5 months post last dose=22% (for RTS,S/AS02 0, 1, 2: 14/44 protected=32% and 4/9 on re-challenge (44%). Overall=14%)
Rampling et al. J Infect Dis 2016 0, 1, 2 months:
12/16 protected at 4 weeks=75%
Re-challenge at 6 months post first CHMI:
5/6 protected=83%
   Overall efficacy at 7 months post last dose=62%
To weight/average: 12.5×5, 22×9, 14×9, 62×6: summed=758.5/29
   Averaged Overall RTS,S 5-8 month efficacy: =26%

Table Legend: Durability of protection after immunisation with R21 and RTS,S malaria vaccines. Overall efficacy at 5-8 months post last dose is calculated by multiplying the proportion protected in the initial challenge by the proportion protected in the late challenge, expressed as a percentage. Only those protected in the initial challenge are re-challenged. R21 durable efficacy appears as about double the rate reported for RTS,S.

In conclusion, this example shows additional useful clinical data on the durability of protection with R21 and also shows that a two dose regimen works in humans.

### Example 5—R21 Low Dose Further Challenge Study

In this example we demonstrate good efficacy in a further challenge study. More specifically, we report a further challenge study of R21/matrix-M vaccines. Key findings are good durability of efficacy at 8.5 months after vaccination that appears better than known RTS,S, and also good efficacy with a two dose low-dose regime.

### Experimental

A further challenge study of R21/matrix-M vaccines was undertaken in September 2017 with two objectives.

The first objective was to evaluate the durability of protection of the 10 microgram (mcg) R21 dose in 50 mcg of matrix-M administered three times at four weekly intervals. In the original challenge study (see examples above-January 2017) nine of eleven vaccinees administered this regimen were sterilely protected. Of the nine protected subjects all were invited for a re-challenge in mid-September 2017 and 5 agreed to participate. Of the five re-challengees three were again sterilely protected and two were not, corresponding to 60% sterile efficacy (see Group 1 in FIG. 13).

FIG. 13 shows outcome of controlled human malaria infection (CHMI) trial in September 2017. In the control group all of almost all the non-vaccinated subjects were infected, in this case 5 of 6. In Group 6 which were two vaccinees immunised 20 months before CHMI with lost dose (10 micrograms) R21 in matrix-M, neither was protected. However, in Group 5 three of five individuals undergoing CHMI initially in late January 2017 (after immunisation in November 2016-early January 2017) were still sterilely protected at 8.5 months after their last immunisation indicating very useful durability of vaccine efficacy. Moreover, four of seven vaccinees receiving just two low 10 microgram doses of R21 one and two months before CHMI in September 2017 were sterilely protected indicating good vaccine efficacy with just two doses of R21 in matrix-M. Also, Group 5 showed that three doses of vaccine with 10, 10 and 2 micrograms generated 72% sterile efficacy (5/6 subjects protected) again showing the efficacy of low doses of R21 in matrix-M. In all vaccinees the dose of matrix-M used was 50 micrograms.

No booster vaccine dose was administered before the re-challenge so that the re-challenge of these subjects occurred about 8.5 months after their last vaccine dose at the start of January. This evidence of durability of sterile protection in most protected subjects out to 8.5 months after last immunisation is very positive and compares favourably with the efficacy of RTS,S/AS01 protection which is generally measured at about 6 months post last dose. Efficacy of R21 at 8.5 month was 49% compared to the reported efficacy of RTS,S/AS vaccine at 5-8 months of 26% (see table below).

The second objective was to assess two new R21 immunisation regimes using low dosages of R21 in matrix-M. In one group (Group 7 in the figure) seven subjects were immunised with just two doses of 10 mcg of R21 in 50 mcg of matrix-M adjuvant. Challenge was at 3 to 4 weeks after the last dose. Of these seven individuals 4 were sterilely protected amounting to 57% vaccine efficacy, apparently the highest efficacy ever reported with a two dose malaria vaccine (from assessment by detailed literature review), showing the technical benefits of the invention.

A further vaccination group received two doses of 10 mcg of R21/matrix-M at a four week interval followed by a 2 mcg R21 dose in 50 mcg matrix-M after a further four weeks. Seven subjects thus immunised were challenged 3-4 weeks later and 5 were sterilely protected, an efficacy rate of 71% (Group 5 in the figure). This is little different from the 9/11 protected with three doses of 10 mcg of R21 in 50 mcg of matrix-M. These data with three low doses of R21/matrix-M provide further evidence of its high level efficacy.

A final group (Group 6 in the figure) were just two vaccinees receiving R21 20 months earlier: neither was sterilely protected. The sample size in this group was very low.

TABLE of DATA: Durability of protection after immunisation with R21 and RTS,S malaria vaccines is shown. Overall efficacy at 5-8 months post last dose is calculated by multiplying the proportion protected in the initial challenge by the proportion protected in the late challenge, expressed as a percentage. Only those protected in the initial challenge are re-challenged. R21 durable efficacy (49%) appears as about double the rate reported for RTS,S (26%).

### Data:

R21 (this Study)
3×10 mcg doses at 0, 1, 2 months challenge at 4 weeks 9/11=82%
5 (of 9) re-challenged at month 8.5 post last dose 3/5=60%
   Overall R21 efficacy at 8.5 months post last dose=49%

### RTS,S (Known/Prior Published Studies)

Regules et al. J Infect Dis 2016 3 week post last dose challenge 10/16=62.5%
5 of 10 re-challenged at 8 months post last dose 1/5=20%
   Overall efficacy at 8.5 months post last dose=12.5%
Kester et al. J Infect Dis 2009 0, 1, 2 months: challenge at 2-3 weeks: 18/36 protected=50%
   Rechallenge at 5.5 months post last dose: 4/9 protected=44%
      Overall efficacy of RTS,S/AS01 at 5.5 months post last dose=22%
(for RTS,S/AS02 0, 1, 2: 14/44 protected=32% and 4/9 on re-challenge (44%). Overall=14%)
Rampling et al. J Infect Dis 2016 0, 1, 2 months: 12/16 protected at 4 weeks=75%
   Re-challenge at 6 months post first CHMI: 5/6 protected=83%
      Overall efficacy at 7 months post last dose=62%
To weight/average: 12.5×5, 22×9, 14×9, 62×6: summed=758.5/29
   Averaged Overall RTS,S 5-8 month efficacy: =26%

### Example 6: R21 Low Dose Vaccination is Immunogenic in West African as Well as UK Subjects

In this example we show good immunogenicity of the low dose regime in West Africa for the first time.

We refer to FIG. 14.

This data demonstrates that R21 low dose vaccination is immunogenic in West African as well as UK subjects. R21 at a dose of 10 micrograms in 50 micrograms matrix-M was administered to both UK subjects (VAC53 trial) and to West African subjects from Burkina Faso (Banfora) (in the Vac060 trial). In each trial vaccination was administered as a three dose regime at months 0, 1 and 2. Immunogenicity after two doses was similar in both populations and only slightly lower in Burkina Faso adults after three doses. These data show good immunogenicity of the low dose R21 vaccine even in an area with high level endemic malaria transmission.

### Example 7: Low Dose Immunogenicity with 2 Micrograms as Well as 10 Micrograms R21

In this example we show that 2 micrograms R21 is also an immunogenic dose.

This demonstrates the utility of low doses-data in earlier examples was on 10 micrograms.

We refer to FIG. 15.

UK healthy adults subjects were immunised with different doses of R21 in a three dose 0, 1, 2 month immunisation regime. The antibody titres induced after three doses of R21 at day 84 were very similar using 2 micrograms, 10 micrograms and 50 micrograms of R21 (in each case in 50 micrograms of matrix-M adjuvant). The local and systemic reactogenicity of the 2 microgram dose regime was better (i.e. reduced) compared to that observed with 10 micrograms or 50 micrograms dosages. Thus we demonstrate a benefit of the low doses taught in the present invention—reduced side effects as illustrated by reduced reactogenicity—yet still achieving antibody titres similar to far higher doses such as 50 microgram doses.

### Example 8: Low Dose Vaccination with R21 Produces Better Durability of Vaccine Responses

Here we surprisingly show that better immune responses are found at day 238 with 10 compared to 50 micrograms R21: i.e. we show that the lower dose is better.

We refer to FIG. 16.

The data demonstrate that low dose vaccination with R21 produces better durability of vaccine responses. UK subjects were immunised at 0, 1, 2 months with R21 with dosages of 10 micrograms or 50 micrograms (always with 50 micrograms matrix-M adjuvant). At day 238 after the first dose antibody levels to the central repeat region of the circumsporozoite protein, a correlate of vaccine efficacy, were higher with the low dose 10 microgram regime than that higher dose 50 microgram regimen i.e. we show that the lower dose is better.

### REFERENCES

1. Efficacy and safety of the RTS,S/AS01 malaria vaccine during 18 months after vaccination: a phase 3 randomized, controlled trial in children and young infants at 11 African sites. PLOS Med, 2014. 11 (7): p. e1001685.
2. Efficacy and safety of RTS,S/AS01 malaria vaccine with or without a booster dose in infants and children in Africa: final results of a phase 3, individually randomised, controlled trial. Lancet, 2015. 386 (9988): p. 31-45.
3. Agnandji, S. T., et al., A phase 3 trial of RTS,S/AS01 malaria vaccine in African infants. N Engl J Med, 2012. 367 (24): p. 2284-95.
4. Agnandji, S. T., et al., First results of phase 3 trial of RTS,S/AS01 malaria vaccine in African children. N Engl J Med, 2011. 365 (20): p. 1863-75.
5. Olotu, A., et al., Seven-Year Efficacy of RTS,S/AS01 Malaria Vaccine among Young African Children. N Engl J Med, 2016. 374 (26): p. 2519-29.
6. Gessner, B. D., D. C. Wraith, and A. Finn, CNS infection safety signal of RTS,S/AS01 and possible association with rabies vaccine. Lancet, 2016. 387 (10026): p. 1376.
7. Klein, S. L., et al., RTS,S Malaria Vaccine and Increased Mortality in Girls. MBio, 2016. 7 (2): p. e00514-16.
8. Malaria Vaccine Funders Group. Malaria Vaccine Technology Roadmap. 2013 20/3/13]; Available from: http://www.who.int/immunization/topics/malaria/vaccine roadmap/TRM_update_nov 13.pdf?ua=1.
9. Collins, K. A., et al., Enhancing protective immunity to malaria with a highly immunogenic virus-like particle vaccine. Sci Rep, 2017. 7: p. 46621.
10. Regules, J. A., et al., Fractional Third and Fourth Dose of RTS,S/AS01 Malaria Candidate Vaccine: A Phase 2a Controlled Human Malaria Parasite Infection and Immunogenicity Study. J Infect Dis, 2016. 214 (5): p. 762-71.
11. Long, C. A. and F. Zavala, Malaria vaccines and human immune responses. Curr Opin Microbiol, 2016. 32: p. 96-102.
12. O'Hara, G. A., et al., Clinical assessment of a recombinant simian adenovirus ChAd63: a potent new vaccine vector. J Infect Dis, 2012. 205 (5): p. 772-81.
13. Kimani, D., et al., Translating the immunogenicity of prime-boost immunization with ChAd63 and MVA ME-TRAP from malaria naive to malaria-endemic populations. Mol Ther, 2014. 22 (11): p. 1992-2003.
14. Ogwang, C., et al., Safety and immunogenicity of heterologous prime-boost immunisation with Plasmodium falciparum malaria candidate vaccines, ChAd63 ME-TRAP and MVA ME-TRAP, in healthy Gambian and Kenyan adults. PLOS One, 2013. 8 (3): p. e57726.
15. Afolabi, M. O., et al., Safety and Immunogenicity of ChAd63 and MVA ME-TRAP in West African Children and Infants. Mol Ther, 2016.
16. Bliss, Viral Vector Malaria Vaccines Induce High-Level T Cell and Antibody Responses in West African Children and Infants. Mol Ther, 2016.
17. Ewer, K. J., et al., Protective CD8+ T-cell immunity to human malaria induced by chimpanzee adenovirus-MVA immunisation. Nat Commun, 2013. 4: p. 2836.
18. Hodgson, S. H., et al., Evaluation of the efficacy of ChAd63-MVA vectored vaccines expressing circumsporozoite protein and ME-TRAP against controlled human malaria infection in malaria-naive individuals. J Infect Dis, 2015. 211 (7): p. 1076-86.
19. Ogwang, C., et al., Prime-boost vaccination with chimpanzee adenovirus and modified vaccinia Ankara encoding TRAP provides partial protection against Plasmodium falciparum infection in Kenyan adults. Sci Transl Med, 2015. 7 (286): p. 286re5.
20. Rampling, T., et al., Safety and High Level Efficacy of the Combination Malaria Vaccine Regimen of RTS,S/AS01B With Chimpanzee Adenovirus 63 and Modified Vaccinia Ankara Vectored Vaccines Expressing ME-TRAP. J Infect Dis, 2016. 214 (5): p. 772-81.
21. Hahn, T., Rapid manufacture and release of a GMP batch of Zaire ebolavirus glycoprotein vaccine made using recombinant baculovirus-Sf9 insect cell culture technology. BioProcess J, 2015. 14 (1): p. 6.
22. Bejon, P., et al., A phase 2b randomised trial of the candidate malaria vaccines FP9 ME-TRAP and MVA ME-TRAP among children in Kenya. PLOS Clin Trials, 2006. 1 (6): p. e29.
23. Rampling, T., et al., Safety and High Level Efficacy of the Combination Malaria Vaccine Regimen of RTS,S/AS01B with ChAd-MVA Vectored Vaccines Expressing ME-TRAP. J Infect Dis, 2016.
24. Lalvani, A., et al., Potent induction of focused Th1-type cellular and humoral immune responses by RTS,S/SBAS2, a recombinant Plasmodium falciparum malaria vaccine. J Infect Dis, 1999. 180 (5): p. 1656-64.
25. Seder, R. A., et al., Protection against malaria by intravenous immunization with a nonreplicating sporozoite vaccine. Science, 2013. 341 (6152): p. 1359-65.
26. Ishizuka, A. S., et al., Protection against malaria at 1 year and immune correlates following PfSPZ vaccination. Nat Med, 2016. 22 (6): p. 614-23.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1-30. (canceled)

31. A method of immunization of a human subject susceptible to Plasmodium falciparum infection comprising administering a composition to said subject, said composition comprising a pharmaceutically acceptable carrier, diluent or excipient and a polypeptide comprising the amino acid sequence of SEQ ID NO: 1, or a sequence having at least 80% sequence identity to the polypeptide SEQ ID NO: 1, wherein said polypeptide is in the form of a virus-like particle (VLP), wherein said VLP comprises less than 10% free hepatitis B surface antigen protein, wherein at least one dose of the polypeptide in the range 1-20 μg is administered to the subject when said subject is at least 18 years old, or at least one dose of the polypeptide in the range of 0.5-10 μg is administered to the subject when said subject is less than 18 years old.

32. The method of claim 31, wherein said VLP comprises a circumsporozoite protein (CSP) sequence and a Hepatitis B surface antigen (HBsAg) sequence in a 1:1 ratio.

33. The method of claim 31, comprising administering at least one dose of 5 μg to 20 μg of said polypeptide per administration when said subject is at least 18 years old, or at least one dose of 2.5 μg to 10 μg of said polypeptide per administration when said subject is less than 18 years old.

34. The method of claim 31, comprising administering at least one dose of 10 μg of said polypeptide per administration when said subject is at least 18 years old, or at least one dose of 5 μg of said polypeptide per administration when said subject is less than 18 years old.

35. The method of claim 34, wherein a final dose contains 10%-50% of the amount of the polypeptide of a first dose.

36. The method of claim 35, wherein said final dose contains 20% of the amount of the polypeptide of said first dose.

37. The method of claim 31, wherein said composition further comprises an adjuvant, wherein said adjuvant comprises: purified saponins obtained from a crude extract of Quillaja saponaria Molina; cholesterol from Lanolin and phospholipid from egg yolk; in a suspension of nano-sized particles in PBS and the polypeptide and said adjuvant are present in a μg:μg ratio in the range 1:1 to 1:50 of polypeptide:adjuvant.

38. The method of claim 37, wherein said ratio is in the range 1:2 to 1:25 of polypeptide:adjuvant.

39. The method of claim 38, wherein said ratio is in the range 1:5 to 1:10 of polypeptide:adjuvant.

40. The method of claim 34, wherein said at least one dose further comprises 10 to 500 μg of an adjuvant when said subject is at least 18 years old, or 5 to 250 μg of said adjuvant when said subject is less than 18 years old, wherein said adjuvant comprises: purified saponins obtained from a crude extract of Quillaja saponaria Molina; cholesterol from Lanolin and phospholipid from egg yolk; in a suspension of nano-sized particles in PBS.

41. The method of claim 34, wherein said at least one dose further comprises 20 to 200 μg of an adjuvant when said subject is at least 18 years old, or 10 to 100 μg of said adjuvant when said subject is less than 18 years old, wherein said adjuvant comprises: purified saponins obtained from a crude extract of Quillaja saponaria Molina; cholesterol from Lanolin and phospholipid from egg yolk; in a suspension of nano-sized particles in PBS.

42. The method of claim 34, wherein said at least one dose further comprises 25 to 50 μg of an adjuvant when said subject is at least 18 years old, or 5 to 50 μg of said adjuvant when said subject is less than 18 years old, wherein said adjuvant comprises: purified saponins obtained from a crude extract of Quillaja saponaria Molina; cholesterol from Lanolin and phospholipid from egg yolk; in a suspension of nano-sized particles in PBS.

43. The method of claim 34, wherein said at least one dose comprises about 10 μg of the polypeptide and about 50 μg adjuvant when said subject is at least 18 years old, or comprises about 5 μg of the polypeptide and about 25 μg adjuvant when said subject is less than 18 years old, wherein said adjuvant comprises purified saponins obtained from a crude extract of Quillaja saponaria Molina; cholesterol from Lanolin and phospholipid from egg yolk; in a suspension of nano-sized particles in PBS.

44. The method of claim 35, wherein said doses are administered to said subject at intervals of 1 week to 12 weeks.

45. The method of claim 35, wherein said doses are administered to said subject at an interval of 4 weeks.

46. The method of claim 31, wherein the composition further comprises at least one of:
(a) a polypeptide comprising the amino acid sequence of SEQ ID NO: 3, or a sequence having at least 80% sequence identity to SEQ ID NO: 3); and
(b) a viral vector, said viral vector comprising a nucleic acid encoding at least one epitope from a malarial antigen, preferably from a P. falciparum or P. vivax antigen.

47. The method of claim 31, wherein said composition is capable of inducing a protective immune response against P. falciparum in said subject.

48. The method of claim 31, wherein said dosage regimen is at least one dose of said polypeptide in the range 0.0000125 to 0.0003333 mg/kg when said subject is at least 18 years old, or 0.00000625 to 0.001667 mg/kg when said subject is less than 18 years old.

49. A composition comprising:
(a) 0.5 μg to 20 μg of a polypeptide comprising the amino acid sequence of SEQ ID NO: 1, or a sequence having at least 80% sequence identity to SEQ ID NO: 1 (R21 polypeptide), wherein said R21 polypeptide is in the form of a virus-like particle (VLP), wherein said VLP comprises less than 10% free hepatitis B surface antigen protein; and
(b) a pharmaceutically acceptable carrier, diluent or excipient.

50. A kit comprising a first composition and a second composition for administration to a human subject:
(a) said first composition comprising 1 μg to 20 μg R21 polypeptide per administration when said subject is at least 18 years old, or 0.5 μg to 10 μg R21 polypeptide per administration when said subject is less than 18 years old, said composition further comprising adjuvant, wherein said adjuvant comprises: purified saponins obtained from a crude extract of Quillaja saponaria Molina; cholesterol from Lanolin and phospholipid from egg yolk; in a suspension of nano-sized particles in PBS; and said adjuvant is present in a ratio in the range 1:1 to 1:50 of R21 polypeptide:adjuvant;
(b) said second composition comprising 10%-50% of the amount of R21 polypeptide of the first composition per administration, said second composition further comprising adjuvant, wherein said adjuvant comprises: purified saponins obtained from a crude extract of Quillaja saponaria Molina; cholesterol from Lanolin and phospholipid from egg yolk; in a suspension of nano-sized particles in PBS; wherein said adjuvant is present in a ratio in the range 1:1 to 1:50 of R21 polypeptide:adjuvant; and
(c) instructions for administration to said subject.


SEQUENCE LISTING (8 sequences)

SEQ ID NO: 1 - protein, 410 aa, synthetic construct
MDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 2 - protein, 414 aa, synthetic construct
MDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEPEA

SEQ ID NO: 3 - protein, 492 aa, synthetic construct
GDRAAGQPAGDRAAGQPAGDRAAGQPAGDRAAGQPAGDRAAGQPAGDRAAGQPAGDRAAGQPAGDRADGQPAGDRADGQPAGNGAGGQAAANGAGNQPGANGAGGQAAANGAGNQPGANGAGGQAAANGAGDQPGANGAGDQPGANGADDQPGANGAGDQPGEDGAGNQPGANGAGDQPGQAAGGNAGGGQGQNNEGANAPNEKSVKEYLDKVRATVGTEWTPCSVTCGVGVRVRRRVNAANKKPEDLTLNDLETDVCTMDKPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 4 - protein, 496 aa, synthetic construct
GDRAAGQPAGDRAAGQPAGDRAAGQPAGDRAAGQPAGDRAAGQPAGDRAAGQPAGDRAAGQPAGDRADGQPAGDRADGQPAGNGAGGQAAANGAGNQPGANGAGGQAAANGAGNQPGANGAGGQAAANGAGDQPGANGAGDQPGANGADDQPGANGAGDQPGEDGAGNQPGANGAGDQPGQAAGGNAGGGQGQNNEGANAPNEKSVKEYLDKVRATVGTEWTPCSVTCGVGVRVRRRVNAANKKPEDLTLNDLETDVCTMDKPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEPEA

SEQ ID NO: 5 - protein, 424 aa, synthetic construct
MMAPDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLGPVTNMENITSGFLGPLLVLQAGFFLLTRILTLPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 6 - protein, 4 aa, synthetic construct
NANP

SEQ ID NO: 7 - protein, 115 aa, synthetic construct
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGI

SEQ ID NO: 8 - protein, 102 aa, synthetic construct
NKNQGNGQGHNMPNDPNRNVDENANANSAVKNNEEPSDKHIKEYLNKIQNSLSTEWSPCSVTCGNGIQVRIKPGSANKPKDELDYANDIEKKICKMEKCSSV